(19) United States
(12) Patent Application Publication
(10) Pub. No.: US 2009/0035238 A1
(43) Pub. Date: Feb. 5, 2009

(54) UV FILTER CAPSULE CONTAINING AN AMINO-SUBSTITUTED HYDROXYBENZOPHENONE

(76) Inventors: Frank Pfluecker, Darmstadt (DE); Hansjuergen Driller, Gross-Umstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/279,173

(22) PCT Filed: Jan. 15, 2007
(86) PCT No.: PCT/EP07/00304

§ 371 Date: Aug. 12, 2008

(30) Foreign Application Priority Data
Feb. 13, 2006 (DE) 10 2006 006 413.5

(51) Int. Cl. A61K 8/11; A61K 8/40; A61Q 17/04; A61K 8/44

(52) U.S. Cl. 424/60; 424/59

(57) ABSTRACT

The present invention relates to UV filter capsules comprising at least one aminosubstituted hydroxybenzophenone, to the use thereof for producing cosmetic or dermatological formulations and preformulations, and to cosmetic or dermatological formulations which comprise the capsules, and to methods for the production thereof.

The present invention relates to UV filter capsules, to the use thereof for the preparation of cosmetic or dermatological formulations or pre-formulations, and to cosmetic or dermatological formulations which comprise the capsules, and to a process for the preparation thereof.

It is generally known that the ultraviolet part of sunlight has a harmful effect on the skin. While rays having a wavelength smaller than 290 nm (so-called UVC region) are absorbed by the ozone layer in the earth's atmosphere, rays in the range between 290 nm and 320 nm, the so-called UVB region, cause erythema, simple sunburn or even various degrees of burning.

It has also been shown that rays in the range between about 320 nm and 400 nm (UVA region) result in damage to the elastic and collagenic fibres of the connective tissue, which causes the skin to age prematurely. Furthermore, these rays are the cause of numerous phototoxic and photoallergic reactions. The harmful influence of UVB radiation may be augmented by UVA radiation.

UVA radiation may furthermore cause skin damage by damaging the skin's own keratin or elastin. This reduces elasticity and water storage capacity of the skin, i.e. the skin becomes less supple and tends to form wrinkles. The strikingly high incidence of skin cancer in localities of strong sunlight shows that damage to erbin formations in the cells is apparently also caused by sunlight, especially by UVA radiation.

However, UV radiation can also result in photochemical reactions, with the photochemical reaction products then engaging in the skin metabolism. Furthermore, UV radiation counts amongst ionising radiation. There is thus a risk of UV exposure also being accompanied by the formation of ionic species, which are then themselves capable of engaging oxidatively in the biochemical processes.

The light-protection filters usual today in cosmetics and dermatology are therefore also divided into UVA and UVB filters. Numerous compounds are known for protection against UVB radiation, mostly derivatives of 3-benzylidenecamphor (for example Eusolex® 6300), of 4-aminobenzoic acid, of cinnamic acid, of salicylic acid, of benzophenone and also of 2-phenylbenzimidazole. For protection against UVA radiation, use is frequently made of dibenzoylmethane derivatives, such as, for example, 4-(tert-butyl)-4′-methoxydibenzoylmethane (Eusolex® 9020) or 4-isopropyldibenzoylmethane (Eusolex® 8020), but these do not have unlimited stability on UV irradiation.

This certain instability to UV radiation is the principal disadvantage of these substances. Under the influence of UV light, 4-(tert-butyl)-4′-methoxy-dibenzoylmethane—representative of all dibenzoylmethane derivatives which absorb in the UV range—has reduced UVA absorption performance. The substance undergoes keto-enol tautomerism. The diketo form absorbs in the UVA region, while the enol form absorbs in the UVC region. On exposure to sunlight or solar simulators, this equilibrium is shifted towards the enol form, which results in lower absorption performance of the UV filter in the UVA region. It is furthermore known that, besides the shift of the keto-enol equilibrium, UV-induced decomposition or photodegradation of Eusolex® 9020 can also occur on exposure.

European Patent Application EP-A-1 046 391 describes amino-substituted hydroxybenzophenones which are suitable as photostable UV-A filters. The compound hexyl 2-(4-diethylamino-2-hydroxybenzoyl)benzoate is commercially available under the trade name Uvinul® A Plus (BASF) as oil-soluble UV-A filter.

Nevertheless, there continues to be a demand for improved administration forms of such compounds which make the use or application of the UV-A filters simpler or safer.

It has now been found that amino-substituted hydroxybenzophenones can be employed extremely well in encapsulated form.

The present invention therefore relates firstly to a UV filter capsule containing at least one amino-substituted hydroxybenzophenone.

Suitable capsules here can have walls of inorganic or organic polymers. For example, U.S. Pat. No. 6,242,099 B1 describes the production of suitable capsules having walls of chitin, chitin derivatives or polyhydroxylated polyamines. Capsules which can particularly preferably be employed in accordance with the invention have walls which can be obtained by a sol-gel process, as described in the applications WO 00/09652, WO 00/72806, WO 00/71084 and WO 03/39510. Preference is in turn given here to capsules whose walls are built up from silica gel (silica; undefined silicon oxide hydroxide). The production of corresponding capsules is known to the person skilled in the art, for example, from the patent applications cited, the relevant content of which expressly also belongs to the subject-matter of the present application.

The process preferably used for the production of the UV filter capsules according to the invention is carried out in three steps:

in step a), an oil-in-water emulsion of the hydrophobic solution comprising the sol-gel precursor, at least one amino-substituted hydroxybenzophenone and optionally at least one cosmetic oil and/or at least one further UVB filter is prepared in an aqueous solution,
in step b), this emulsion from step a) is mixed with another aqueous solution in order to accelerate the condensation polymerisation reaction, and
in step c), reaction products are optionally separated off from the sol-gel precursor.

After an appropriate reaction time, in which the mixture can also be warmed or cooled or the pH can also be modified, the capsules formed can be isolated by means which are familiar to the person skilled in the art. For example, they can be centrifuged or filtered. A particularly preferred type of isolation is spray drying.

In general, a suspension comprising the UV filter capsules according to the invention in a form as can be employed directly in cosmetic or dermatological compositions is obtained after step c). Re-suspension of the isolated capsules in, for example, deionised water or in another medium is also conceivable and can be used for use in the compositions according to the invention.

The hydrophobic solution from step a) and also the aqueous solutions from steps a) and b) may comprise surfactants and/or other additives which may improve this process and/or the product.

The sol-gel precursor can be a metal or semi-metal alkoxide monomer, a metal ester, semi-metal ester or a partially hydrolysed and partially condensed polymer, or a mixture thereof.

Suitable and preferred sol-gel precursors are also compounds of the formula M(R)ₙ(P)ₘ, in which M denotes a metal or semi-metal, preferably Si, R denotes a hydrolysable substituent, and n denotes an integer from 2 to 4, P denotes an unpolymerisable substituent, and m denotes an integer from 0 to 4, or a partially hydrolysed or partially condensed polymer thereof, or any mixture thereof.

The process described above is particularly preferably carried out using tetraethyl orthosilicate or a partially hydrolysed or partially condensed polymer thereof, or a mixture thereof. Tetraethyl orthosilicate is very particularly preferably employed as sol-gel precursor.

Further details are disclosed in the working examples.

In detail, the encapsulation gives rise to the following advantages:
The hydrophilicity of the capsule wall can be adjusted independently of the solubility of the UV filter. Thus, for example, it is also possible to incorporate hydrophobic UV filters into purely aqueous formulations. In addition, the oily impression on application of the composition comprising hydrophobic UV filters, which is frequently perceived as unpleasant, is suppressed.
The skin penetration by organic UV filters and the associated irritation potential on direct application to the human skin is repeatedly discussed in the literature. The encapsulation of the corresponding substances which is proposed here suppresses this effect.
In general, encapsulation of individual UV filters or other ingredients enables formulation problems arising due to the interaction of individual formulation constituents with one another, such as crystallisation processes, precipitation and agglomerate formation, to be avoided since the interaction is suppressed.
The encapsulation of amino-substituted hydroxybenzophenone derivatives enables a significantly higher UVA content in a capsule to be achieved than with other commercially available UVA filters (for example dibenzoylmethane derivatives). In particular in aqueous formulations, improved UV-A protection can thus be provided.
The high UVA absorber content in the capsules gives rise to excellent light-protection properties for day-care products, which are intended to provide continuous protection against UV-A radiation.

It is advantageous here for the capsules to be sufficiently small that they cannot be seen with the naked eye. In order to achieve the above-mentioned effects, it is furthermore necessary for the capsules to be sufficiently stable and only to release the encapsulated active compound (UV filter) to the environment to a slight extent, or not at all.

The amino-substituted hydroxybenzophenone derivatives employed in accordance with the invention are preferably compounds of the formula I:

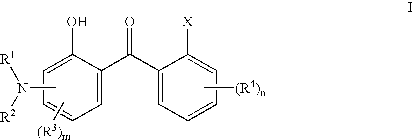

in which the variables, independently of one another, have the following meaning:
R¹ and R² hydrogen, C₁-C₂₀-alkyl, C₂-C₁₀-alkenyl, C₃-C₁₀-cycloalkyl, C₃-C₁₀-cycloalkenyl, where the substituents R¹ and R² can form a 5- or 6-membered ring together with the nitrogen atom to which they are bonded;
R³ and R⁴ C₁-C₂₀-alkyl, C₂-C₁₀-alkenyl, C₃-C₁₀-cycloalkyl, C₃-C₁₀-cycloalkenyl, C₁-C₁₂-alkoxy, C₁-C₂₀-alkoxy, C₁-C₂₀-alkoxycarbonyl, C₁-C₁₂-alkylamino, C₁-C₁₂-dialkylamino, aryl, heteroaryl, optionally substituted, water-solubilising substituents selected from the group consisting of a nitrile group, carboxylate, sulfonate or ammonium radicals;
X hydrogen, COOR⁵, CONR⁶R⁷;
R⁵ to R⁷ hydrogen, C₁-C₂₀-alkyl, C₂-C₁₀-alkenyl, C₃-C₁₀-cycloalkyl, C₃-C₁₀-cycloalkenyl, —(Y—O)ₒ-Z, aryl;
Y —(CH₂)₂—, —(CH₂)₄—, —CH(CH₃)—CH₂—;
Z —CH₂—CH₃, —CH₂—CH₂-CH₃, —CH₂—CH₂-CH₂—CH₃, —CH/(CH₃)—CH₃;
m 0 to 3;
n 0 to 4;
o 1 to 20.

The corresponding substance class, the preparation of the compounds and the properties thereof are described in EP-A-1 046 391.

Alkyl radicals R¹ to R⁷ which may be mentioned are branched or unbranched C₁-C₂₀-alkyl chains, preferably methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, 2-ethylhexyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl.

Alkenyl radicals R¹ to R⁷ which may be mentioned are branched or unbranched C₂-C₁₀-alkenyl chains, preferably vinyl, propenyl, isopropenyl, 1-butenyl, 2-butenyl, 1-pentenyl, 2-pentenyl, 2-methyl-1-butenyl, 2-methyl-2-butenyl, 3-methyl-1-butenyl, 1-hexenyl, 2-hexenyl, 1-heptenyl, 2-heptenyl, 1-octenyl or 2-octenyl.

Cycloalkyl radicals which may be mentioned for R¹ to R⁷ are preferably branched or unbranched C₃-C₁₀-cycloalkyl chains, such as cyclopropyl, cyclobutyl, cyclopentyl, cyclohexyl, cycloheptyl, 1-methylcyclopropyl, 1-ethylcyclopropyl, 1-propylcyclopropyl, 1-butylcyclopropyl, 1-pentylcyclo-propyl, 1-methyl-1-butylcyclopropyl, 1,2-dimethylcyclopropyl, 1-methyl-2-ethylcyclopropyl, cyclooctyl, cyclononyl or cyclodecyl.

Cycloalkenyl radicals which may be mentioned for R¹ to R⁷ are preferably branched or unbranched C₃-C₁₀-cycloalkenyl chains having one or more double bonds, such as cyclopropenyl, cyclobutenyl, cyclopentenyl, cyclopentadienyl, cyclohexenyl, 1,3-cyclohexadienyl, 1,4-cyclohexadienyl, cycloheptenyl, cycloheptatrienyl, cyclooctenyl, 1,5-cyclooctadienyl, cyclooctatetraenyl, cyclononenyl or cyclodecyl.

The cycloalkenyl and cycloalkyl radicals may optionally be substituted by one or more, for example 1 to 3, radicals, such as halogen, for example fluorine, chlorine or bromine, cyano, nitro, amino, C₁-C₄-alkylamino, C₁-C₄-dialkylamino, hydroxyl, C₁-C₄-alkyl, C₁-C₄-alkoxy or other radicals, or may contain 1 to 3 heteroatoms, such as sulfur, nitrogen, whose free valences may be saturated by hydrogen or C₁-C₄-alkyl, or oxygen in the ring.

Alkoxy radicals which come into consideration for R³ and R⁴ are those having 1 to 12 C atoms, preferably having 1 to 8 C atoms.

For example, the following may be mentioned:

| | |
|---|---|
| methoxy | ethoxy |
| isopropoxy | n-propoxy |
| 1-methylpropoxy | n-butoxy |
| n-pentoxy | 2-methylpropoxy |
| 3-methylbutoxy | 1,1-dimethylpropoxy |
| 2,2-dimethylpropoxy | hexoxy |
| 1-methyl-1-ethylpropoxy | heptoxy |
| octoxy | 2-ethyhexoxy |

Alkoxycarbonyl radicals for R³ and R⁴ are, for example, esters which contain the above-mentioned alkoxy radicals or radicals of higher alcohols, for example having up to 20 C atoms, such as iso-C₁₋₅-alcohol.

Mono- or dialkylamino radicals which come into consideration for R³ and R⁴ are those which contain alkyl radicals having 1 to 12 C atoms, such as, for example, methyl, n-propyl, n-butyl, 2-methylpropyl, 1,1-dimethylpropyl, hexyl, heptyl, 2-ethylhexyl, isopropyl, 1-methylpropyl, n-pentyl, 3-methylbutyl, 2,2-dimethylpropyl, 1-methyl-1-ethylpropyl and octyl.

Aryl is taken to mean aromatic rings or ring systems having 6 to 18 carbon atoms in the ring system, for example phenyl or naphthyl, each of which may optionally be substituted by one or more radicals, such as halogen, for example fluorine, chlorine or bromine, cyano, nitro, amino, C₁-C₄-alkylamino, C₁-C₄-dialkylamino, hydroxyl, C₁-C₄-alkyl, C₁-C₄-alkoxy or other radicals. Preference is given to optionally substituted phenyl, methoxyphenyl and naphthyl.

Heteroaryl radicals are advantageously simple or condensed aromatic ring systems having one or more heteroaromatic 3- to 7-membered rings. Heteroatoms which may be present in the ring or ring system are one or more nitrogen, sulfur and/or oxygen atoms.

Hydrophilic radicals, i.e. those which facilitate the water solubility of the compounds of the formula I, for R³ and R⁴ are, for example, the nitrile group and carboxyl and sulfoxy radicals and in particular salts thereof with any desired physiologically tolerated cations, such as the alkali-metal salts or such as the trialkylammonium salts, such as tri(hydroxyalkyl)ammonium salts or 2-methylpropan-1-ol-2-ammonium salts. Furthermore, ammonium radicals, in particular alkylammonium radicals, with any desired physiologically tolerated anions come into consideration.

The substituents R¹ and R² can form a 5- or 6-membered ring, for example a pyrrolidine or piperidine ring, together with the nitrogen atom to which they are bonded.

The amino group can be in the ortho, meta or para position relative to the carbonyl group. The para position is preferred.

Preference is given to compounds of the formula Ib

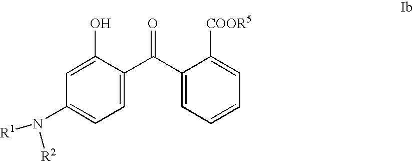

in which the substituents, independently of one another, have the following meaning:
R¹ and R² hydrogen, C₁-C₁₂-alkyl, where the substituents R¹ and R² can form a 5- or 6-membered ring together with the nitrogen atom to which they are bonded;
R⁵ hydrogen, C₁-C₁₂-alkyl, C₃-C₆-cycloalkyl.

Alkyl radicals R¹, R² and R⁵ which may be mentioned are branched or unbranched C₁-C₁₂-alkyl chains, preferably methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl or 2-ethylhexyl.

Particularly preferred alkyl radicals which may be mentioned for R¹, R² and R⁵ are methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 2-ethylhexyl.

C₃-C₆-cycloalkyl radicals which may be mentioned for R⁵ are particularly preferably cyclopropyl, cyclopentyl and cyclohexyl.

Furthermore, compounds of the formula Ib in which the substituents R¹, R² and R⁵ have the combination mentioned in the following table have particular photostable properties.

Ib

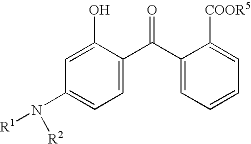

| R¹ | R² | R⁵ |
|---|---|---|
| H | H | H |
| H | H | Methyl |
| H | H | Ethyl |
| H | H | n-Propyl |
| H | H | 1-Methylethyl |
| H | H | n-Butyl |
| H | H | 1-Methylpropyl |
| H | H | 2-Methylpropyl |
| H | H | 1,1-Dimethylethyl |
| H | H | n-Pentyl |
| H | H | 1-Methylbutyl |
| H | H | 2-Methylbutyl |
| H | H | 2,2-Dimethylpropyl |
| H | H | 2-Ethylhexyl |
| H | H | Cyclopropyl |
| H | H | Cyclopentyl |
| H | H | Cyclohexyl |
| Methyl | Methyl | H |
| Methyl | Methyl | Methyl |
| Methyl | Methyl | Ethyl |
| Methyl | Methyl | n-Propyl |
| Methyl | Methyl | 1-Methylethyl |
| Methyl | Methyl | n-Butyl |
| Methyl | Methyl | 1-Methylpropyl |
| Methyl | Methyl | 2-Methylpropyl |
| Methyl | Methyl | 1,1-Dimethylethyl |
| Methyl | Methyl | n-Pentyl |
| Methyl | Methyl | 1-Methylbutyl |
| Methyl | Methyl | 2-Methylbutyl |
| Methyl | Methyl | 2,2-Dimethylpropyl |
| Methyl | Methyl | 2-Ethylhexyl |
| Methyl | Methyl | Cyclopropyl |
| Methyl | Methyl | Cyclopentyl |
| Methyl | Methyl | Cyclohexyl |
| Ethyl | Ethyl | H |
| Ethyl | Ethyl | Methyl |
| Ethyl | Ethyl | Ethyl |
| Ethyl | Ethyl | n-Propyl |
| Ethyl | Ethyl | 1-Methylethyl |
| Ethyl | Ethyl | n-Butyl |
| Ethyl | Ethyl | 1-Methylpropyl |
| Ethyl | Ethyl | 2-Methylpropyl |
| Ethyl | Ethyl | 1,1-Dimethylethyl |
| Ethyl | Ethyl | n-Pentyl |
| Ethyl | Ethyl | 1-Methylbutyl |
| Ethyl | Ethyl | 2-Methylbutyl |
| Ethyl | Ethyl | 2,2-Dimethylpropyl |
| Ethyl | Ethyl | 2-Ethylhexyl |
| Ethyl | Ethyl | Cyclopropyl |
| Ethyl | Ethyl | Cyclopentyl |
| Ethyl | Ethyl | Cyclohexyl |
| n-Propyl | n-Propyl | H |
| n-Propyl | n-Propyl | Methyl |
| n-Propyl | n-Propyl | Ethyl |
| n-Propyl | n-Propyl | n-Propyl |
| n-Propyl | n-Propyl | 1-Methylethyl |
| n-Propyl | n-Propyl | n-Butyl |
| n-Propyl | n-Propyl | 1-Methylpropyl |
| n-Propyl | n-Propyl | 2-Methylpropyl |
| n-Propyl | n-Propyl | 1,1-Dimethylethyl |
| n-Propyl | n-Propyl | n-Pentyl |
| n-Propyl | n-Propyl | 1-Methylbutyl |
| n-Propyl | n-Propyl | 2-Methylbutyl |
| n-Propyl | n-Propyl | 2,2-Dimethylpropyl |
| n-Propyl | n-Propyl | 2-Ethylhexyl |
| n-Propyl | n-Propyl | Cyclopropyl |
| n-Propyl | n-Propyl | Cyclopentyl |
| n-Propyl | n-Propyl | Cyclohexyl |
| 1 -Methylethyl | 1 -Methylethyl | H |
| 1 -Methylethyl | 1 -Methylethyl | Methyl |
| 1 -Methylethyl | 1 -Methylethyl | Ethyl |
| 1 -Methylethyl | 1 -Methylethyl | n-Propyl |
| 1 -Methylethyl | 1 -Methylethyl | 1-Methylethyl |
| 1 -Methylethyl | 1 -Methylethyl | n-Butyl |
| 1 -Methylethyl | 1 -Methylethyl | 1-Methylpropyl |
| 1 -Methylethyl | 1 -Methylethyl | 2-Methylpropyl |
| 1 -Methylethyl | 1 -Methylethyl | 1,1-Dimethylethyl |
| 1 -Methylethyl | 1 -Methylethyl | n-Pentyl |
| 1 -Methylethyl | 1 -Methylethyl | 1-Methylbutyl |
| 1 -Methylethyl | 1 -Methylethyl | 2-Methylbutyl |
| 1 -Methylethyl | 1 -Methylethyl | 2,2-Dimethylpropyl |
| 1 -Methylethyl | 1 -Methylethyl | 2-Ethylhexyl |
| 1 -Methylethyl | 1 -Methylethyl | Cyclopropyl |
| 1 -Methylethyl | 1 -Methylethyl | Cyclopentyl |
| 1 -Methylethyl | 1 -Methylethyl | Cyclohexyl |
| n-Butyl | n-Butyl | H |
| n-Butyl | n-Butyl | Methyl |
| n-Butyl | n-Butyl | Ethyl |
| n-Butyl | n-Butyl | n-Propyl |
| n-Butyl | n-Butyl | 1-Methylethyl |
| n-Butyl | n-Butyl | n-Butyl |
| n-Butyl | n-Butyl | 1-Methylpropyl |
| n-Butyl | n-Butyl | 2-Methylpropyl |
| n-Butyl | n-Butyl | 1,1-Dimethylethyl |
| n-Butyl | n-Butyl | n-Pentyl |
| n-Butyl | n-Butyl | 1-Methylbutyl |
| n-Butyl | n-Butyl | 2-Methylbutyl |
| n-Butyl | n-Butyl | 2,2-Dimethylpropyl |
| n-Butyl | n-Butyl | 2-Ethylhexyl |
| n-Butyl | n-Butyl | Cyclopropyl |
| n-Butyl | n-Butyl | Cyclopentyl |
| n-Butyl | n-Butyl | Cyclohexyl |
| 1-Methylpropyl | 1-Methylpropyl | H |
| 1-Methylpropyl | 1-Methylpropyl | Methyl |
| 1-Methylpropyl | 1-Methylpropyl | Ethyl |
| 1-Methylpropyl | 1-Methylpropyl | n-Propyl |
| 1-Methylpropyl | 1-Methylpropyl | 1-Methylethyl |
| 1-Methylpropyl | 1-Methylpropyl | n-Butyl |
| 1-Methylpropyl | 1-Methylpropyl | 1-Methylpropyl |
| 1-Methylpropyl | 1-Methylpropyl | 2-Methylpropyl |
| 1-Methylpropyl | 1-Methylpropyl | 1,1-Dimethylethyl |
| 1-Methylpropyl | 1-Methylpropyl | n-Pentyl |
| 1-Methylpropyl | 1-Methylpropyl | 1-Methylbutyl |
| 1-Methylpropyl | 1-Methylpropyl | 2-Methylbutyl |
| 1-Methylpropyl | 1-Methylpropyl | 2,2-Dimethylpropyl |
| 1-Methylpropyl | 1-Methylpropyl | 2-Ethylhexyl |
| 1-Methylpropyl | 1-Methylpropyl | Cyclopropyl |
| 1-Methylpropyl | 1-Methylpropyl | Cyclopentyl |
| 1-Methylpropyl | 1-Methylpropyl | Cyclohexyl |
| 2-Methylpropyl | 2-Methylpropyl | H |
| 2-Methylpropyl | 2-Methylpropyl | Methyl |
| 2-Methylpropyl | 2-Methylpropyl | Ethyl |
| 2-Methylpropyl | 2-Methylpropyl | n-Propyl |
| 2-Methylpropyl | 2-Methylpropyl | 1-Methylethyl |
| 2-Methylpropyl | 2-Methylpropyl | n-Butyl |
| 2-Methylpropyl | 2-Methylpropyl | 1-Methylpropyl |
| 2-Methylpropyl | 2-Methylpropyl | 2-Methylpropyl |
| 2-Methylpropyl | 2-Methylpropyl | 1,1-Dimethylethyl |
| 2-Methylpropyl | 2-Methylpropyl | n-Pentyl |
| 2-Methylpropyl | 2-Methylpropyl | 1-Methylbutyl |
| 2-Methylpropyl | 2-Methylpropyl | 2-Methylbutyl |
| 2-Methylpropyl | 2-Methylpropyl | 2,2-Dimethylpropyl |
| 2-Methylpropyl | 2-Methylpropyl | 2-Ethylhexyl |
| 2-Methylpropyl | 2-Methylpropyl | Cyclopropyl |
| 2-Methylpropyl | 2-Methylpropyl | Cyclopentyl |
| 2-Methylpropyl | 2-Methylpropyl | Cyclohexyl |
| 1,1-Dimethylethyl | 1,1-Dimethylethyl | H |
| 1,1-Dimethylethyl | 1,1-Dimethylethyl | Methyl |
| 1,1-Dimethylethyl | 1,1-Dimethylethyl | Ethyl |
| 1,1-Dimethylethyl | 1,1-Dimethylethyl | n-Propyl |
| 1,1-Dimethylethyl | 1,1-Dimethylethyl | 1-Methylethyl |
| 1,1-Dimethylethyl | 1,1-Dimethylethyl | n-Butyl |
| 1,1-Dimethylethyl | 1,1-Dimethylethyl | 1-Methylpropyl |
| 1,1-Dimethylethyl | 1,1-Dimethylethyl | 2-Methylpropyl |
| 1,1-Dimethylethyl | 1,1-Dimethylethyl | 1,1-Dimethylethyl |
| 1,1-Dimethylethyl | 1,1-Dimethylethyl | n-Pentyl |
| 1,1-Dimethylethyl | 1,1-Dimethylethyl | 1-Methylbutyl |
| 1,1-Dimethylethyl | 1,1-Dimethylethyl | 2-Methylbutyl |
| 1,1-Dimethylethyl | 1,1-Dimethylethyl | 2,2-Dimethylpropyl |
| 1,1-Dimethylethyl | 1,1-Dimethylethyl | 2-Ethylhexyl |
| 1,1-Dimethylethyl | 1,1-Dimethylethyl | Cyclopropyl |
| 1,1-Dimethylethyl | 1,1-Dimethylethyl | Cyclopentyl |
| 1,1-Dimethylethyl | 1,1-Dimethylethyl | Cyclohexyl |
| n-Pentyl | n-Pentyl | H |
| n-Pentyl | n-Pentyl | Methyl |
| n-Pentyl | n-Pentyl | Ethyl |
| n-Pentyl | n-Pentyl | n-Propyl |
| n-Pentyl | n-Pentyl | 1-Methylethyl |
| n-Pentyl | n-Pentyl | n-Butyl |
| n-Pentyl | n-Pentyl | 1-Methylpropyl |
| n-Pentyl | n-Pentyl | 2-Methylpropyl |
| n-Pentyl | n-Pentyl | 1,1-Dimethylethyl |
| n-Pentyl | n-Pentyl | n-Pentyl |
| n-Pentyl | n-Pentyl | 1-Methylbutyl |
| n-Pentyl | n-Pentyl | 2-Methylbutyl |
| n-Pentyl | n-Pentyl | 2,2-Dimethylpropyl |
| n-Pentyl | n-Pentyl | 2-Ethylhexyl |
| n-Pentyl | n-Pentyl | Cyclopropyl |
| n-Pentyl | n-Pentyl | Cyclopentyl |
| n-Pentyl | n-Pentyl | Cyclohexyl |
| 1-Methylbutyl | 1-Methylbutyl | H |
| 1-Methylbutyl | 1-Methylbutyl | Methyl |
| 1-Methylbutyl | 1-Methylbutyl | Ethyl |
| 1-Methylbutyl | 1-Methylbutyl | n-Propyl |
| 1-Methylbutyl | 1-Methylbutyl | 1-Methylethyl |
| 1-Methylbutyl | 1-Methylbutyl | n-Butyl |
| 1-Methylbutyl | 1-Methylbutyl | 1-Methylpropyl |
| 1-Methylbutyl | 1-Methylbutyl | 2-Methylpropyl |
| 1-Methylbutyl | 1-Methylbutyl | 1,1-Dimethylethyl |
| 1-Methylbutyl | 1-Methylbutyl | n-Pentyl |
| 1-Methylbutyl | 1-Methylbutyl | 1-Methylbutyl |
| 1-Methylbutyl | 1-Methylbutyl | 2-Methylbutyl |
| 1-Methylbutyl | 1-Methylbutyl | 2,2-Dimethyipropyl |
| 1-Methylbutyl | 1-Methylbutyl | 2-Ethyhexyl |
| 1-Methylbutyl | 1-Methylbutyl | Cyclopropyl |
| 1-Methylbutyl | 1-Methylbutyl | Cyclopentyl |
| 1-Methylbutyl | 1-Methylbutyl | Cyclohexyl |
| 2-Methylbutyl | 2-Methylbutyl | H |
| 2-Methylbutyl | 2-Methylbutyl | Methyl |
| 2-Methylbutyl | 2-Methylbutyl | Ethyl |
| 2-Methylbutyl | 2-Methylbutyl | n-Propyl |
| 2-Methylbutyl | 2-Methylbutyl | 1-Methylethyl |
| 2-Methylbutyl | 2-Methylbutyl | n-Butyl |
| 2-Methylbutyl | 2-Methylbutyl | 1-Methylpropyl |
| 2-Methylbutyl | 2-Methylbutyl | 2-Methylpropyl |
| 2-Methylbutyl | 2-Methylbutyl | 1,1-Dimethylethyl |
| 2-Methylbutyl | 2-Methylbutyl | n-Pentyl |
| 2-Methylbutyl | 2-Methylbutyl | 1-Methylbutyl |
| 2-Methylbutyl | 2-Methylbutyl | 2-Methylbutyl |
| 2-Methylbutyl | 2-Methylbutyl | 2,2-Dimethylpropyl |
| 2-Methylbutyl | 2-Methylbutyl | 2-Ethylhexyl |
| 2-Methylbutyl | 2-Methylbutyl | Cyclopropyl |
| 2-Methylbutyl | 2-Methylbutyl | Cyclopentyl |
| 2-Methylbutyl | 2-Methylbutyl | Cyclohexyl |
| 2,2-Dimethylpropyl | 2,2-Dimethylpropyl | H |
| 2,2-Dimethylpropyl | 2,2-Dimethylpropyl | Methyl |
| 2,2-Dimethylpropyl | 2,2-Dimethylpropyl | Ethyl |
| 2,2-Dimethylpropyl | 2,2-Dimethylpropyl | n-Propyl |
| 2,2-Dimethylpropyl | 2,2-Dimethylpropyl | 1-Methylethyl |
| 2,2-Dimethylpropyl | 2,2-Dimethylpropyl | n-Butyl |
| 2,2-Dimethylpropyl | 2,2-Dimethylpropyl | 1-Methylpropyl |
| 2,2-Dimethylpropyl | 2,2-Dimethylpropyl | 2-Methylpropyl |
| 2,2-Dimethylpropyl | 2,2-Dimethylpropyl | 1,1-Dimethylethyl |
| 2,2-Dimethylpropyl | 2,2-Dimethylpropyl | n-Pentyl |
| 2,2-Dimethylpropyl | 2,2-Dimethylpropyl | 1-Methylbutyl |
| 2,2-Dimethylpropyl | 2,2-Dimethylpropyl | 2-Methylbutyl |
| 2,2-Dimethylpropyl | 2,2-Dimethylpropyl | 2,2-Dimethylpropyl |
| 2,2-Dimethylpropyl | 2,2-Dimethylpropyl | 2-Ethylhexyl |
| 2,2-Dimethylpropyl | 2,2-Dimethylpropyl | Cyclopropyl |
| 2,2-Dimethylpropyl | 2,2-Dimethylpropyl | Cyclopentyl |
| 2,2-Dimethylpropyl | 2,2-Dimethylpropyl | Cyclohexyl |
| 2-Ethylhexyl | 2-Ethylhexyl | H |
| 2-Ethylhexyl | 2-Ethylhexyl | Methyl |
| 2-Ethylhexyl | 2-Ethylhexyl | Ethyl |
| 2-Ethylhexyl | 2-Ethylhexyl | n-Propyl |
| 2-Ethylhexyl | 2-Ethylhexyl | 1-Methylethyl |
| 2-Ethylhexyl | 2-Ethylhexyl | n-Butyl |
| 2-Ethylhexyl | 2-Ethylhexyl | 1-Methylpropyl |
| 2-Ethylhexyl | 2-Ethylhexyl | 2-Methylpropyl |
| 2-Ethylhexyl | 2-Ethylhexyl | 1,1-Dimethylethyl |
| 2-Ethylhexyl | 2-Ethylhexyl | n-Pentyl |
| 2-Ethylhexyl | 2-Ethylhexyl | 1-Methylbutyl |
| 2-Ethylhexyl | 2-Ethylhexyl | 2-Methylbutyl |
| 2-Ethylhexyl | 2-Ethylhexyl | 2,2-Dimethylpropyl |
| 2-Ethylhexyl | 2-Ethylhexyl | 2-Ethylhexyl |
| 2-Ethylhexyl | 2-Ethylhexyl | Cyclopropyl |
| 2-Ethylhexyl | 2-Ethylhexyl | Cyclopentyl |
| 2-Ethylhexyl | 2-Ethylhexyl | Cyclohexyl |
| Cyclopropyl | Cyclopropyl | H |
| Cyclopropyl | Cyclopropyl | Methyl |
| Cyclopropyl | Cyclopropyl | Ethyl |
| Cyclopropyl | Cyclopropyl | n-Propyl |
| Cyclopropyl | Cyclopropyl | 1-Methylethyl |
| Cyclopropyl | Cyclopropyl | n-Butyl |
| Cyclopropyl | Cyclopropyl | 1-Methylpropyl |
| Cyclopropyl | Cyclopropyl | 2-Methylpropyl |
| Cyclopropyl | Cyclopropyl | 1,1-Dimethylethyl |
| Cyclopropyl | Cyclopropyl | n-Pentyl |
| Cyclopropyl | Cyclopropyl | 1-Methylbutyl |
| Cyclopropyl | Cyclopropyl | 2-Methylbutyl |
| Cyclopropyl | Cyclopropyl | 2,2-Dimethylpropyl |
| Cyclopropyl | Cyclopropyl | 2-Ethylhexyl |
| Cyclopropyl | Cyclopropyl | Cyclopropyl |
| Cyclopropyl | Cyclopropyl | Cyclopentyl |
| Cyclopropyl | Cyclopropyl | Cyclohexyl |
| Cyclopentyl | Cyclopentyl | H |
| Cyclopentyl | Cyclopentyl | Methyl |
| Cyclopentyl | Cyclopentyl | Ethyl |
| Cyclopentyl | Cyclopentyl | n-Propyl |
| Cyclopentyl | Cyclopentyl | 1-Methylethyl |
| Cyclopentyl | Cyclopentyl | n-Butyl |
| Cyclopentyl | Cyclopentyl | 1-Methylpropyl |
| Cyclopentyl | Cyclopentyl | 2-Methylpropyl |
| Cyclopentyl | Cyclopentyl | 1,1-Dimethylethyl |
| Cyclopentyl | Cyclopentyl | n-Pentyl |
| Cyclopentyl | Cyclopentyl | 1-Methylbutyl |
| Cyclopentyl | Cyclopentyl | 2-Methylbutyl |
| Cyclopentyl | Cyclopentyl | 2,2-Dimethylpropyl |
| Cyclopentyl | Cyclopentyl | 2-Ethylhexyl |
| Cyclopentyl | Cyclopentyl | Cyclopropyl |
| Cyclopentyl | Cyclopentyl | Cyclopentyl |
| Cyclopentyl | Cyclopentyl | Cyclohexyl |
| Cyclohexyl | Cyclohexyl | H |
| Cyclohexyl | Cyclohexyl | Methyl |
| Cyclohexyl | Cyclohexyl | Ethyl |
| Cyclohexyl | Cyclohexyl | n-Propyl |
| Cyclohexyl | Cyclohexyl | 1-Methylethyl |
| Cyclohexyl | Cyclohexyl | n-Butyl |
| Cyclohexyl | Cyclohexyl | 1-Methylpropyl |
| Cyclohexyl | Cyclohexyl | 2-Methylpropyl |
| Cyclohexyl | Cyclohexyl | 1,1-Dimethylethyl |
| Cyclohexyl | Cyclohexyl | n-Pentyl |
| Cyclohexyl | Cyclohexyl | 1-Methylbutyl |
| Cyclohexyl | Cyclohexyl | 2-Methylbutyl |
| Cyclohexyl | Cyclohexyl | 2,2-Dimethylpropyl |
| Cyclohexyl | Cyclohexyl | 2-Ethylhexyl |
| Cyclohexyl | Cyclohexyl | Cyclopropyl |
| Cyclohexyl | Cyclohexyl | Cyclopentyl |
| Cyclohexyl | Cyclohexyl | Cyclohexyl |

The invention also relates to amino-substituted hydroxybenzophenones of the formula Ic

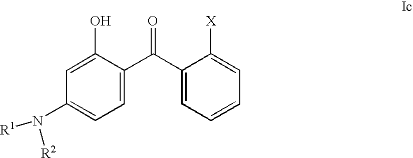

in which the variables, independently of one another, have the following meaning:
R¹ and R² hydrogen, C₁-C₈-alkyl, where the substituents R¹ and R² can form a 5- or 6-membered ring together with the nitrogen atom to which they are bonded;
X COOR⁵, CONR⁶R⁷;
R⁵ C₂-C₁₂-alkyl, C₅-C₆-cycloalkyl;
R⁶ and R⁷ hydrogen, C₁-C₁₂-alkyl, C₅-C₆-cycloalkyl.

Alkyl radicals R¹ and R² which may be mentioned are branched or unbranched C₁-C₈-alkyl chains, preferably methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl or 2-ethylhexyl.

Alkyl radicals R⁵ which may be mentioned are branched or unbranched C₂-C₁₂-alkyl chains, preferably ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, 2-ethylhexyl, n-nonyl, n-decyl, n-undecyl or n-dodecyl.

Alkyl radicals R⁶ and R⁷ which may be mentioned are branched or unbranched C₂-C₁₂-alkyl chains, preferably methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl or 2-ethylhexyl.

Cycloalkyl radicals which may be mentioned for R⁵ to R⁷ are preferably branched or unbranched C₅-C₆-cycloalkyl chains, such as cyclopentyl or cyclohexyl.

Preference is given to compounds of the formula Ic in which, independently of one another, R¹ and R² denote C₁-C₄-alkyl, R⁵ denotes C₃-C₈-alkyl and R⁶ and R⁷ denote C₁-C₈-alkyl.

Particular preference is given to compounds of the formula Ic in which, independently of one another, R¹ and R² denote ethyl, R⁵ denotes C₅-C₈-alkyl and R⁶ and R⁷ denote C₁-C₈-alkyl from the respective lists of substituents above.

Especial preference is given in accordance with the invention to the use of hexyl 2-(4-diethylamino-2-hydroxybenzoyl)benzoate.

The compounds of the formula I to be used in accordance with the invention are either commercially available or can be prepared as described in EP-A-1 046 391.

The UV filter capsules here usually consist of 1 to 99% by weight, preferably 5 to 55% by weight and particularly preferably 20 to 40% by weight, of amino-substituted hydroxybenzophenone.

Besides the amino-substituted hydroxybenzophenone, the capsule preferably also contains at least one cosmetic oil. This oil is preferably a conventional cosmetic oil or a hydrophobic UV filter which is liquid at room temperature. The choice of correspondingly suitable cosmetic oils via the solubility of the hydroxybenzophenone compound selected and optionally the desired absorption spectrum presents the person skilled in the art with no difficulties.

The following oils can particularly preferably be used here (INCI names): Octocrylene, Ethylhexyl Methoxycinnamate, Dibutyl Adipate, C12-15 Alkyl Benzoate, C12-13 Alkyl Lactate, Propylene Glycol Dicaprylate/Dicaprate, Diethylhexyl Adipate, PEG-7 Glyceryl Cocoate, Caprylic/Capric Triglyceride, Ethylhexyl Ethylhexanoate, Isopropyl Alcohol, PPG-3 Myristyl Ether, D1-C12-13 Alkyl Tartrate, Ethanol, Hexyl Laurate, PEG-7 Hydrogenated Castor Oil, Di-C12-13 Alkyl Malate, Isopropyl Stearate.

In a preferred embodiment of the present invention, the oil is an ester of saturated and/or unsaturated, branched and/or unbranched alkanecarboxylic acids having a chain length of 3 to 30 C atoms and saturated and/or unsaturated, branched and/or unbranched alkylene glycols having a chain length of 2 to 30 C atoms.

In an embodiment which is preferred in accordance with the invention, the capsules, besides the UV-A filter, also contain at least one UV-B filter, which is preferably selected from methoxycinnamic acid derivatives, salicylic acid derivatives or diphenylacrylate derivatives.

Suitable methoxycinnamic acid derivatives are, for example, esters, such as ethylhexyl methoxycinnamate (for example Eusolex® 2292), isopentyl 4-methoxycinnamate, for example as a mixture of the isomers (for example Neo Heliopan® E 1000), and mixtures thereof.

Suitable salicylic acid derivatives are, for example, 2-ethylhexyl salicylate (for example Eusolex® OS), 4-isopropylbenzyl salicylate (for example Megasol®) or 3,3,5-trimethylcyclohexyl salicylate (for example Eusolex® HMS).

Of the diphenylacrylate derivatives, 2-ethylhexyl 2-cyano-3,3-diphenylacrylate (for example Eusolex® OCR; octocrylene) is particularly preferred.

It is preferred here for the UV filter capsule to contain amino-substituted hydroxybenzophenone and UV-B filters in the percent by weight ratio 10:1 to 1:3, preferably in the percent by weight ratio 5:1 to 1:2.

Particular preference is given in accordance with the invention to the following capsule compositions:
A) capsules containing 5 to 42% by weight of amino-substituted hydroxybenzophenone and 58 to 95% by weight of cinnamic acid derivative;
B) capsules containing 5 to 44% by weight of amino-substituted hydroxybenzophenone and 56 to 95% by weight of diphenylacrylate derivative;
C) capsules containing 50 to 90% by weight of amino-substituted hydroxybenzophenone and 10 to 50% by weight of salicylic acid derivative.

The capsules having a high UV-A filter content are particularly suitable for use in the area of day care.

Particular preference is given here to the use of capsules which contain
A) amino-substituted hydroxybenzophenone to cinnamic acid derivative in a percent by weight ratio>1:1.5, preferably in a percent by weight ratio of about 1:1; or
B) amino-substituted hydroxybenzophenone to diphenylacrylate derivative in a percent by weight ratio<4:1, preferably in a percent by weight ratio of about 3:1.

Further preferred combinations are disclosed in the claims.

The present invention furthermore relates to the use of the capsules for the preparation of a cosmetic or dermatological composition having light-protection properties and formulations comprising at least one amino-substituted hydroxybenzophenone and at least one vehicle which is suitable for topical purposes, characterised in that at least part of the amino-substituted hydroxybenzophenones is in the form of a capsule according to the invention.

The composition may include or comprise, essentially consist of or consist of the said requisite or optional constituents or ingredients. All compounds or components which can be used in the compositions are either known and commercially available or can be synthesised by known processes or processes described here.

For the purposes of the invention, the term composition is used synonymously with the term formulation.

In particular, the formulations can be an aqueous dispersion of the capsules which preferably comprises 5 to 80% by weight of capsules, particularly preferably 30 to 50% by weight of capsules.

The compositions can be pre-dispersions which are on the one hand themselves directly suitable as cosmetic or dermatological composition and on the other hand can simplify the preparation of such compositions. The present application therefore likewise relates to the corresponding use of the formulations for the preparation of a cosmetic or dermatological composition having light-protection properties.

Corresponding formulations can preferably be aqueous compositions, in particular gels, or oil-in-water emulsions (O/W emulsions).

In accordance with the invention, the preparation of cosmetic or dermatological compositions comprising capsules according to the invention can therefore be carried out in various ways:
One process according to the invention for the preparation of a cosmetic or dermatological composition having light-protection properties is characterised in that a capsule according to the invention is mixed with further ingredients.
A further process according to the invention for the preparation of a cosmetic or dermatological composition having light-protection properties is characterised in that the formulation described above is emulsified with an oil if the cosmetic or dermatological composition is an oil-in-water emulsion (O/W emulsion).

The capsules are preferably present here in formulations according to the invention in such amounts which ensure that the encapsulated UV filters are present in effective amounts in the formulation.

In a preferred variant of the present invention, the formulation according to the invention consists of the said ingredients, i.e. the formulation is the above-mentioned pre-formulation.

The present invention furthermore relates to the use of a formulation according to the invention for the preparation of a cosmetic or dermatological composition having light-protection properties.

The cosmetic or dermatological composition having light-protection properties can exist in various forms. Thus, it can be, for example, a solution, an emulsion or microemulsion of the water-in-oil (W/O) type or of the oil-in-water (O/W) type, a multiple emulsion, for example of the water-in-oil-in-water (W/O/W) type, a gel, a solid stick, an ointment or also an aerosol.

It is particularly preferred here for the cosmetic or dermatological composition to be an aqueous composition, in particular a gel, or an emulsion, in particular an oil-in-water emulsion (O/W emulsion), since the advantages of the formulations according to the invention come to bear particularly well in the preparation of such compositions.

The present invention accordingly furthermore also relates to a process for the preparation of a cosmetic or dermatological composition having light-protection properties, characterised in that a formulation according to the invention is mixed with further ingredients. An oil phase is particularly preferably emulsified in the formulation, and an oil-in-water emulsion (O/W emulsion) is thus prepared.

The present invention therefore furthermore relates to emulsions comprising the formulation according to the invention described above in or as the water phase. Particular preference is given here to oil-in-water emulsions (O/W emulsions).

Emulsions according to the invention are advantageous and comprise, for example, the said fats, oils, waxes and other fatty substances, as well as water and an emulsifier, as usually used for a composition of this type.

The lipid phase may advantageously be selected from the following group of substances:
mineral oils, mineral waxes;
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of the esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, from the group of the esters of aromatic carboxylic acids and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group of the branched and unbranched hydrocarbons and waxes, silicone oils, dialkyl ethers, the group of the saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24 C atoms, in particular 12-18 C atoms. The fatty acid triglycerides may advantageously be selected, for example, from the group of the synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The oil phase is advantageously selected from the group 2-ethylhexyl isostearate, octyldodecanol, isotridecyl isononanoate, isoeicosene, 2-ethylhexyl cocoate, C₁₂₋₁₅-alkyl benzoate, caprylic/capric acid triglyceride, dicapryl ether.

Particularly advantageous are mixtures of C₁₂₋₁₅-alkyl benzoate and 2-ethylhexyl isostearate, mixtures of C₁₂₋₁₅-alkyl benzoate and isotridecyl isononanoate, as well as mixtures of C₁₂₋₁₅-alkyl benzoate, 2-ethylhexyl isostearate and isotridecyl isononanoate.

Of the hydrocarbons, paraffin oil, squalane and squalene may advantageously be used for the purposes of the present invention.

Furthermore, the oil phase may also advantageously have a content of cyclic or linear silicone oils or consist entirely of oils of this type, although it is preferred to use an additional content of other oil-phase components in addition to the silicone oil or the silicone oils.

The silicone oil to be used in accordance with the invention is advantageously cyclomethicone (octamethylcyclotetrasiloxane). However, it is also advantageous for the purposes of the present invention to use other silicone oils, for example hexamethylcyclotrisiloxane, polydimethylsiloxane, poly(methylphenylsiloxane).

Also particularly advantageous are mixtures of cyclomethicone and isotridecyl isononanoate and of cyclomethicone and 2-ethylhexyl isostearate.

The aqueous phase of the compositions according to the invention optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

Emulsions according to the invention are advantageous and comprise, for example, the said fats, oils, waxes and other fatty substances, as well as water and an emulsifier, as usually used for a formulation of this type.

Emulsifiers that can be used are, for example, the known W/O and O/W emulsifiers. It is advantageous to use further conventional co-emulsifiers in the preferred O/W emulsions according to the invention.

Co-emulsifiers which are advantageous in accordance with the invention are, for example, O/W emulsifiers, principally from the group of the sub-stances having HLB values of 11-16, very particularly advantageously having HLB values of 14.5-15.5, so long as the O/W emulsifiers have saturated radicals R and R′. If the O/W emulsifiers have unsaturated radicals R and/or R′ or in the case of isoalkyl derivatives, the preferred HLB value of such emulsifiers may also be lower or higher.

It is advantageous to select the fatty alcohol ethoxylates from the group of the ethoxylated stearyl alcohols, cetyl alcohols, cetylstearyl alcohols (cetearyl alcohols). Particular preference is given to the following: polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19), polyethylene glycol (20) cetylstearyl ether (ceteareth-20).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laureth-14 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favourable to select the sorbitan esters from the group c polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

The following can be employed as optional W/O emulsifiers, but ones which may nevertheless be advantageous in accordance with the invention:

fatty alcohols having 8 to 30 C atoms, monoglycerol esters of saturated and/or unsaturated, branched and/or unbranched alkanecarboxylic acids having a chain length of 8 to 24 C atoms, in particular 12-18 C atoms, diglycerol esters of saturated and/or unsaturated, branched and/or unbranched alkanecarboxylic acids having a chain length of from 8 to 24 C atoms, in particular 12-18 C atoms, monoglycerol ethers of saturated and/or unsaturated, branched and/or unbranched alcohols having a chain length of 8 to 24 C atoms, in particular 12-18 C atoms, diglycerol ethers of saturated and/or unsaturated, branched and/or unbranched alcohols having a chain length of 8 to 24 C atoms, in particular 12-18 C atoms, propylene glycol esters of saturated and/or unsaturated, branched and/or unbranched alkanecarboxylic acids having a chain length of 8 to 24 C atoms, in particular 12-18 C atoms, and sorbitan esters of saturated and/or unsaturated, branched and/or unbranched alkanecarboxylic acids having a chain length of 8 to 24 C atoms, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate or PEG 30 dipolyhydroxystearate.

Cosmetic and dermatological compositions to be prepared in accordance with the invention additionally advantageously, although not necessarily, comprise inorganic pigments based on metal oxides and/or other metal compounds which are sparingly soluble or insoluble in water, in particular the oxides of titanium (TiO₂), zinc (ZnO), iron (for example Fe₂O₃), zirconium (ZrO₂), silicon (SiO₂), manganese (for example MnO), aluminium (Al₂O₃), cerium (for example Ce₂O₃), mixed oxides of the corresponding metals and mixtures of such oxides. Particular preference is given to pigments based on TiO₂ and in particular micronised TiO₂.

In accordance with the invention, the cosmetic and/or dermatological light-protection formulations can have the usual composition and serve for cosmetic and/or dermatological light protection, furthermore for the treatment, care and cleansing of the skin and/or of the hair and as make-up product in decorative cosmetics.

Particular preference is given in accordance with the invention to the preparation of cosmetic and dermatological compositions which are in the form of a sunscreen. These may advantageously additionally comprise at least one further UVA filter and/or at least one further UVB filter and/or at least one inorganic pigment, preferably hydrophobic inorganic micropigments.

Particular preference is given to UV filters whose physiological acceptability has already been demonstrated. For both UVA and UVB filters, there are substances which are known from the specialist literature, for example

benzylidenecamphor derivatives, such as 3-(4′-methylbenzylidene)dl-camphor (for example Eusolex® 6300), 3-benzylidenecamphor (for example Mexoryl® SD), polymers of N-{(2 and 4)-[(2-oxoborn-3-ylidene)methyl]-benzyl}acrylamide (for example Mexoryl® SW), N,N,N-trimethyl-4-(2-oxo-born-3-ylidenemethyl)anilinium methylsulfate (for example Mexoryl® SK) or (2-oxoborn-3-ylidene)toluene-4-sulfonic acid (for example Mexoryl® SL),

benzoyl- or dibenzoylmethanes, such as 1-(4-tert-butylphenyl)-3-(4-methoxyphenyl)propane-1,3-dione (for example Eusolex® 9020) or 4-isopropyldibenzoylmethane (for example Eusolex® 8020),

benzophenones, such as 2-hydroxy-4-methoxybenzophenone (for example Eusolex® 4360) or 2-hydroxy-4-methoxybenzophenone-5-sulfonic acid and the sodium salt thereof (for example Uvinul® MS-40),

methoxycinnamic acid esters, such as octyl methoxycinnamate (for example Eusolex® 2292) or isopentyl 4-methoxycinnamate, for example as a mixture of the isomers (for example Neo Heliopan® E 1000),

salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS),

4-aminobenzoic acid and derivatives, such as 4-aminobenzoic acid, 2-ethylhexyl 4-(dimethylamino)benzoate (for example Eusolex® 6007) or ethoxylated ethyl 4-aminobenzoate (for example Uvinul® P25),

phenylbenzimidazolesulfonic acids, such as 2-phenylbenzimidazole-5-sulfonic acid and the potassium, sodium and triethanolamine salts thereof (for example Eusolex® 232), 2,2-(1,4-phenylene)bisbenzimidazole-4,6-disulfonic acid or salts thereof (for example Neoheliopan® AP) or 2,2-(1,4-phenylene)bisbenzimidazole-6-sulfonic acid;

and further substances, such as
2-ethylhexyl 2-cyano-3,3-diphenylacrylate (for example Eusolex® OCR),
3,3′-(1,4-phenylenedimethylene)bis(7,7-dimethyl-2-oxobicyclo[2.2.1]hept-1-ylmethanesulfonic acid and salts thereof (for example Mexoryl® SX) and
2,4,6-trianilino-(p-carbo-2′-ethylhexyl-1′-oxy)-1,3,5-triazine (for example Uvinul® T 150)
hexyl 2-(4-diethylamino-2-hydroxybenzoyl)benzoate (for example Uvinul® UVA Plus, BASF).

The compounds mentioned in the list should only be regarded as examples. It is of course also possible to use other UV filters.

These organic UV filters are generally incorporated into cosmetic formulations in an amount of 0.5 to 10 percent by weight, preferably 1-8% by weight.

Further suitable organic UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®),
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),
dimethicone diethylbenzal malonate (CAS No. 207 574-74-1) or
2,2′-methylenebis(6-(2H-benzotriazol-2-yl)-4-(1,1,3,3-tetramethylbutyl)phenol) (CAS No. 103 597-45-1)
2,2′-(1,4-phenylene)bis(1H-benzimidazole-4,6-disulfonic acid, monosodium salt) (CAS No. 180 898-37-7) and
2,4-bis{[4-(2-ethylhexyloxy)-2-hydroxy]phenyl}-6-(4-methoxyphenyl)-1,3,5-triazine (CAS No. 103 597-45-, 187 393-00-6).

Further suitable UV filters are also methoxyflavones corresponding to German patent application DE 10232595.

Organic UV filters are generally incorporated into cosmetic formulations in an amount of from 0.5 to 20 percent by weight, preferably 1-15%.

Conceivable inorganic UV filters are those from the group of the titanium dioxides, such as, for example, coated titanium dioxide (for example Eusolex® T-2000, Eusolex® T-AQUA, Eusolex® T-AVO), zinc oxides (for example Sachtotec®), iron oxides or also cerium oxides. These inorganic UV filters are generally incorporated into cosmetic compositions in an amount of 0.5 to 20 percent by weight, preferably 2-10% by weight.

Preferred compounds having UV-filtering properties are 3-(4′-methylbenzylidene)dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, and the potassium, sodium and triethanolamine salts thereof.

All the organic UV filters mentioned can also be employed in encapsulated form, in which case the encapsulatioon techniques described above can be employed. In accordance with the invention, the UV light-protection filters described here can in each case be used alone or naturally also in combination, which is preferred, in sunscreens. They can be combined with UV-B/A chromophores, for example all filters approved and known worldwide, for improving the protective performance (SPF boost) through synergistic effects. They can preferably be employed in combination both with inorganic and with organic UV-A and UV-B filters or mixtures thereof.

Besides the compounds described here, the compositions according to the invention may also comprise at least one photostabiliser, preferably conforming to the formula I

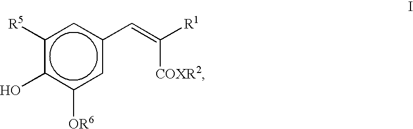

where
R¹ is selected from —C(O)CH₃, —CO₂R³, —C(O)NH₂ and —C(O)N(R⁴)₂;
X is O or NH,
R² is a linear or branched C₁₋₃₀-alkyl radical;
R³ is a linear or branched C₁₂₀-alkyl radical,
all R⁴, independently of one another, are H or linear or branched C₁₋₈-alkyl radicals,
R⁵ is H, a linear or branched C₁₋₈-alkyl radical or a linear or branched —O—C₁₋₈-alkyl radical, and
R⁶ is a C₁₋₈-alkyl radical,
   where the photostabiliser is particularly preferably bis(2-ethylhexyl) 2-(4-hydroxy-3,5-dimethoxybenzylidene)malonate. Corresponding photostabilisers and their preparation and use are described in International patent application WO 03/007906, the disclosure content of which expressly also belongs to the subject-matter of the present application.

It is furthermore possible and advantageous to combine the compositions according to the invention with antioxidants. A combination of this type then exhibits both a protective action as antioxidant and also against burns due to UV radiation. A protective action against oxidative stress or against the action of free radicals can thus also be achieved.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Mixtures of antioxidants are likewise suitable for use in the cosmetic compositions according to the invention. Known and commercial mixtures are, for example, mixtures comprising, as active ingredients, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004).

The compositions according to the invention may comprise vitamins as further ingredients. The cosmetic compositions according to the invention preferably comprise vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active ingredient), thiamine (vitamin B₁), nicotinic acid (niacin), pyridoxine, pyridoxal, pyridoxamine (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin.

The compositions according to the invention may in addition comprise further conventional skin-protecting or skin-care active ingredients. These may in principle be any active ingredients known to the person skilled in the art, such as, in particular, flavone derivatives, chromone derivatives, compatible solutes and other active ingredients.

It may be preferred for the composition according to the invention to comprise at least one repellent, where the repellent is preferably selected from N,N-diethyl-3-methylbenzamide, ethyl 3-(acetylbutylamino)propionate, di-methyl phthalate, butopyronoxyl, 2,3,4,5-bis(2-butylene)tetrahydro-2-furaldehyde, N,N-diethylcaprylamide, N,N-diethylbenzamide, o-chloro-N,N-diethylbenzamide, dimethyl carbate, di-n-propyl isocinchomeronate, 2-ethylhexane-1,3-diol, N-octylbicycloheptenedicarboximide, piperonyl butoxide, 1-(2-methylpropoxycarbonyl)-2-(hydroxyethyl)piperidine, or mixtures thereof, where it is particularly preferably selected from N,N-diethyl-3-methylbenzamide, ethyl 3-(acetylbutylamino)propionate, 1-(2-methylpropoxycarbonyl)-2-(hydroxyethyl)piperidine, or mixtures thereof.

The compositions according to the invention which comprise repellents are preferably insect repellents. Insect repellents are available in the form of solutions, gels, sticks, rollers, pump sprays and aerosol sprays, with solutions and sprays forming the majority of the commercially available products. The basis for these two product forms is usually formed by alcoholic or aqueous/alcoholic solutions with addition of fatting substances and slight perfuming.

In accordance with the invention, flavone derivatives is taken to mean flavonoids and coumaranones. In accordance with the invention, flavonoids is taken to mean the glycosides of flavonones, flavones, 3-hydroxyflavones (=flavonols), aurones, isoflavones and rotenoids [Römpp Chemie Lexikon [Römpp's Lexicon of Chemistry], Volume 9, 1993]. For the purposes of the present invention, however, this term is also taken to mean the aglycones, i.e. the sugar-free constituents, and the derivatives of the flavonoids and aglycones. For the purposes of the present invention, the term flavonoid is furthermore also taken to mean anthocyanidine (cyanidine). For the purposes of the present invention, the term coumaranones is also taken to mean derivatives thereof.

Preferred flavonoids are derived from flavonones, flavones, 3-hydroxyflavones, aurones and isoflavones, in particular from flavonones, flavones, 3-hydroxyflavones and aurones.

The flavonoids are preferably selected from the following compounds: 4,6,3′,4′-tetrahydroxyaurone, quercetin, rutin, isoquercetin, eriodictyol, taxifolin, luteolin, trishydroxyethylquercetin (troxequercetin), trishydroxyethylrutin (troxerutin), trishydroxyethylisoquercetin (troxeisoquercetin), trishydroxyethylluteolin (troxeluteolin), α-glycosylrutin, tiliroside and the sulfates and phosphates thereof. Of the flavonoids, particular preference is given, as active substances according to the invention, to rutin, tiliroside, α-glycosylrutin and troxerutin.

Of the phenols having an antioxidative action, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I. M. C. M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′,4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A. E. M. F. Soffers, I. M. C. M. Rietjens; Free Radical Biology & Medicine 2001, 31(7), 869-881, have investigated the pH dependence of the antioxidant action of hydroxyflavones. Quercetin exhibits the greatest activity amongst the structures investigated over the entire pH range.

Suitable antioxidants are furthermore compounds of the formula II

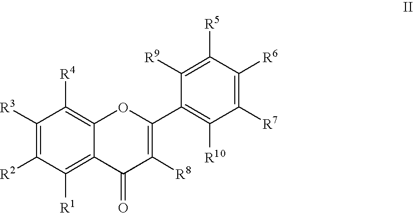

where R¹ to R¹⁰ may be identical or different and are selected from
H
OR¹¹
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom in the chain and furthermore the alkyl chain may also be interrupted by oxygen, and/or
C₃- to C₁₋₁₀-cycloalkyl groups and/or C₃- to C₁₂-cycloalkenyl groups, where the rings may each also be bridged by —(CH₂)ₙ— groups, where n=1 to 3,
where all OR¹¹ are, independently of one another,
   OH
   straight-chain or branched C₁- to C₂₀-alkoxy groups,
   straight-chain or branched C₃- to C₂₀-alkenyloxy groups,
   straight-chain or branched C₁- to C₂₀-hydroxyalkoxy groups, where the hydroxyl group(s) may be bonded to a primary or secondary carbon atom in the chain and furthermore the alkyl chain may also be interrupted by oxygen, and/or
   C₃- to C₁₋₁₀-cycloalkoxy groups and/or C₃- to C₁₂-cycloalkenyloxy groups, where the rings may each also be bridged by —(CH₂)ₙ— groups, where n=1 to 3, and/or
   mono- and/or oligoglycosyl radicals,
with the proviso that at least 4 radicals from R¹ to R⁷ are OH and that at least 2 pairs of adjacent —OH groups are present in the molecule,
or R², R⁵ and R⁶ are OH and the radicals R¹, R³, R⁴ and R⁷⁻¹⁰ are H,
   as described in German patent application DE-A-10244282.

Of the coumaranones, preference is given to 4,6,3′,4′-tetrahydroxybenzyl-coumaranone-3.

The term chromone derivatives is preferably taken to mean certain chromen-2-one derivatives which are suitable as active ingredients for the preventative treatment of human skin and human hair against ageing processes and harmful environmental influences. At the same time, they exhibit a low irritation potential for the skin, have a positive effect on water binding in the skin, maintain or increase the elasticity of the skin and thus promote smoothing of the skin. These compounds preferably conform to the formula III

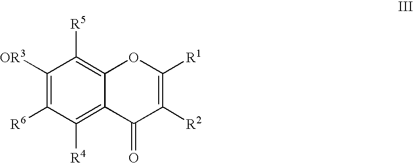

where
R¹ and R² may be identical or different and are selected from
H, —C(═O)—R⁷, —C(═O)—OR⁷,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom in the chain and furthermore the alkyl chain may also be interrupted by oxygen, and/or
C₃- to C₁₋₁₀-cycloalkyl groups and/or C₃- to C₁₂-cycloalkenyl groups, where the rings may each also be bridged by —(CH₂)ₙ— groups, where n=1 to 3,
   R³ is H or straight-chain or branched C₁- to C₂₀-alkyl groups,

### R⁴ is H or OR⁸,

R⁵ and R⁶ may be identical or different and are selected from
—H, —OH,
straight-chain or branched C₁- to C₂₀-alkyl groups,
straight-chain or branched C₃- to C₂₀-alkenyl groups and
straight-chain or branched C₁- to C₂₀-hydroxyalkyl groups, where the hydroxyl group may be bonded to a primary or secondary carbon atom in the chain and furthermore the alkyl chain may also be interrupted by oxygen, and
   R⁷ is H, straight-chain or branched C₁- to C₂₀-alkyl groups, a polyhydroxyl compound, such as preferably an ascorbic acid radical or glycosidic radicals, and
   R⁸ is H or straight-chain or branched C₁- to C₂₀-alkyl groups, where at least two of the substituents R¹, R² and R⁴—R⁶ are not H or at least one substituent from R¹ and R² is —C(═O)—R⁷ or —C(═O)—OR⁷.

The proportion of one or more compounds selected from flavonoids, chromone derivatives and coumaranones in the composition according to the invention is preferably from 0.001 to 5% by weight, particularly preferably from 0.01 to 2% by weight, based on the composition as a whole.

Particularly preferred active ingredients are, for example, also so-called compatible solutes. These are substances which are involved in the osmoregulation of plants or microorganisms and can be isolated from these organisms. The generic term compatible solutes here also encompasses the osmolytes described in German patent application DE-A-10133202. Suitable osmolytes are, for example, the polyols, methylamine compounds and amino acids and respective precursors thereof. Osmolytes in the sense of German patent application DE-A-10133202 are, in particular, substances from the group consisting of the polyols, such as, for example, myo-inositol, mannitol or sorbitol, and/or one or more of the osmolytically active substances mentioned below:

taurine, choline, betaine, phosphorylcholine, glycerophosphorylcholines, glutamine, glycine, α-alanine, glutamate, aspartate and proline. Precursors of these substances are, for example, glucose, glucose polymers, phosphatidylcholine, phosphatidylinositol, inorganic phosphates, proteins, peptides and polyamino acids. Precursors are, for example, compounds which are converted into osmolytes by metabolic steps.

Compatible solutes which are preferably employed in accordance with the invention are substances selected from the group consisting of pyrimidinecarboxylic acids (such as ectoine and hydroxyectoine), proline, betaine, glutamine, cyclic diphosphoglycerate, N-acetylornithine, trimethylamine N-oxide, di-myo-inositol phosphate (DIP), cyclic 2,3-diphosphoglycerate (cDPG), 1,1-diglycerol phosphate (DGP), β-mannosyl glycerate (firoin), β-mannosyl glyceramide (firoin-A) and/or dimannosyl diinositol phosphate (DMIP) or an optical isomer, derivative, for example an acid, a salt or ester, of these compounds, or combinations thereof.

Of the pyrimidinecarboxylic acids, particular mention should be made here of ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid) and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoine and ectoine derivatives, such as hydroxyectoine, can advantageously be used in medicaments. In particular, hydroxyectoine can be employed for the preparation of a medicament for the treatment of skin diseases. Other areas of application of hydroxyectoine and other ectoine derivatives are typically in areas in which, for example, trehalose is used as additive. Thus, ectoine derivatives, such as hydroxyectoine, can be used as protectant in dried yeast and bacterial cells. Pharmaceutical products, such as non-glycosylated, pharmaceutically active peptides and proteins, for example t-PA, can also be protected with ectoine or its derivatives.

Of the cosmetic applications, particular mention should be made of the use of ectoine and ectoine derivatives for the care of aged, dry or irritated skin. Thus, European patent application EP-A-0 671 161 describes, in particular, that ectoine and hydroxyectoine are employed in cosmetic compositions, such as powders, soaps, surfactant-containing cleansing products, lipsticks, rouge, make-up, care creams and sunscreen preparations.

Preference is given here to the use of a pyrimidinecarboxylic acid of the following formula IV

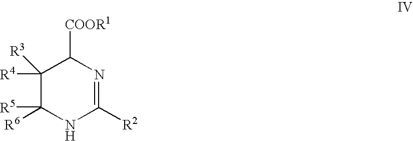

in which R¹ is a radical H or C₁₋₈-alkyl, R² is a radical H or C₁₋₄-alkyl, and R³, R⁴, R⁵ and R⁶ are each, independently of one another, a radical from the group consisting of H, OH, NH₂ and C₁₋₄-alkyl. Preference is given to the use of pyrimidinecarboxylic acids in which R² is a methyl or ethyl group, and R¹ or R⁵ and R⁶ are H. Particular preference is given to the use of the pyrimidinecarboxylic acids ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid). In this case, the compositions according to the invention preferably comprise pyrimidinecarboxylic acids of this type in amounts of up to 15% by weight.

It is particularly preferred in accordance with the invention for the compatible solutes to be selected from di-myo-inositol phosphate (DIP), cyclic 2,3-diphosphoglycerate (cDPG), 1,1-diglycerol phosphate (DGP), β-mannosyl glycerate (firoin), β-mannosyl glyceramide (firoin-A) and/or dimannosyl diinositol phosphate (DMIP), ectoine, hydroxyectoine or mixtures thereof.

Of the aryl oximes likewise preferably employed, preference is given to the use of 2-hydroxy-5-methyllaurophenone oxime, which is also known as HMLO, LPO or F5. Its suitability for use in cosmetic compositions is dis-closed, for example, in DE-A-41 16 123. Compositions which comprise 2-hydroxy-5-methyllaurophenone oxime are accordingly suitable for the treatment of skin diseases which are accompanied by inflammation. It is known that compositions of this type can be used, for example, for the therapy of psoriasis, various forms of eczema, irritative and toxic dermatitis, UV dermatitis and further allergic and/or inflammatory diseases of the skin and integumentary appendages. Compositions according to the invention which comprise an aryl oxime, preferably 2-hydroxy-5-methyllaurophenone oxime, exhibit surprising antiinflammatory suitability. The compositions here preferably comprise from 0.01 to 10% by weight of the aryl oxime, it being particularly preferred for the composition to comprise from 0.05 to 5% by weight of aryl oxime.

In a further, likewise preferred embodiment of the present invention, the composition according to the invention comprises at least one self-tanning agent.

Advantageous self-tanning agents which can be employed are, inter alia:

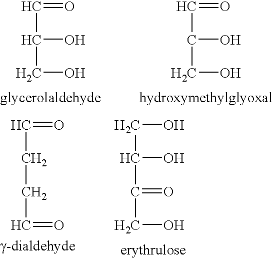

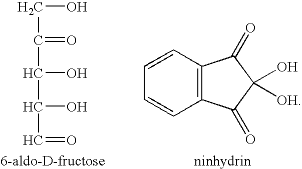

Mention should also be made of 5-hydroxy-1,4-naphthoquinone (juglone), which can be extracted from the shells of fresh walnuts, and 2-hydroxy-1,4-naphthoquinone (lawsone), which occurs in henna leaves. The flavonoid diosmetin and glycosides or sulfates thereof can also be employed. These compounds can be employed in the form of pure substances or plant extracts. Diosmetin can preferably be employed, for example, in the form of a chrysanthemum extract.

Very particular preference is given to 1,3-dihydroxyacetone (DHA), a trifunctional sugar which occurs in the human body, and derivatives thereof

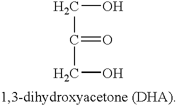

The said self-tanning agents can be employed alone or in the form of a mixture. It is particularly preferred here for DHA to be employed in a mixture with a further self-tanning agent of those mentioned above.

Furthermore, the compositions according to the invention may also comprise dyes and coloured pigments. The dyes and coloured pigments can be selected from the corresponding positive list in the German Cosmetics Regulation or the EU list of cosmetic colorants. In most cases, they are identical with the dyes approved for foods. Advantageous coloured pigments are, for example, titanium dioxide, mica, iron oxides (for example Fe₂O₃, Fe₃O₄, FeO(OH)) and/or tin oxide. Advantageous dyes are, for example, carmine, Berlin Blue, Chromium Oxide Green, Ultramarine Blue and/or Manganese Violet. It is particularly advantageous to select the dyes and/or coloured pigments from the following list. The Colour Index numbers (CINs) are taken from the Rowe Colour Index, 3rd Edition, Society of Dyers and Colourists, Bradford, England, 1971.

| Chemical or other name | CIN | Colour |
|---|---|---|
| Pigment Green | 10006 | Green |
| Acid Green 1 | 10020 | Green |
| 2,4-dinitrohydroxynaphthalene-7-sulfonic acid | 10316 | Yellow |
| Pigment Yellow 1 | 11680 | Yellow |
| Pigment Yellow 3 | 11710 | Yellow |
| Pigment Orange 1 | 11725 | Orange |
| 2,4-dihydroxyazobenzene | 11920 | Orange |
| Solvent Red 3 | 12010 | Red |
| 1-(2′-chloro-4′-nitro-1′-phenylazo)-2-hydroxynaphthalene | 12085 | Red |
| Pigment Red 3 | 12120 | Red |
| Ceres Red; Sudan Red; Fat Red G | 12150 | Red |
| Pigment Red 112 | 12370 | Red |
| Pigment Red 7 | 12420 | Red |
| Pigment Brown 1 | 12480 | Brown |
| N-(5-chloro-2,4-dimethoxyphenyl)-4-[[5-[(diethylamino)- | 12490 | Red |
| sulfonyl]-2-methoxyphenyl]azo]-3-hydroxynaphthalene-2- | | |
| carboxamide | | |
| Disperse Yellow 16 | 12700 | Yellow |
| 1-(4-Sulfo-1-phenylazo)-4-aminobenzene-5-sulfonic acid | 13015 | Yellow |
| 2,4-dihydroxyazobenzene-4′-sulfonic acid | 14270 | Orange |
| 2-(2,4-dimethylphenylazo-5-sulfonyl)-1-hydroxynaphthalene-4- | 14700 | Red |
| sulfonic acid | | |
| 2-(4-Sulfo-1-naphthylazo)-1-naphthol-4-sulfonic acid | 14720 | Red |
| 2-(6-Sulfo-2,4-xylylazo)-1-naphthol-5-sulfonic acid | 14815 | Red |
| 1-(4′-Sulfophenylazo)-2-hydroxynaphthalene | 15510 | Orange |
| 1-(2-Sulfonyl-4-chloro-5-carboxy-1-phenylazo)-2-hydroxy- | 15525 | Red |
| naphthalene | | |
| 1-(3-Methylphenylazo-4-sulfonyl)-2-hydroxynaphthalene | 15580 | Red |
| 1-(4′,(8′)-Sulfonylnaphthylazo)-2-hydroxynaphthalene | 15620 | Red |
| 2-Hydroxy-1,2′-azonaphthalene-1′-sulfonic acid | 15630 | Red |
| 3-Hydroxy-4-phenylazo-2-naphthylcarboxylic acid | 15800 | Red |
| 1-(2-Sulfo-4-methyl-1-phenylazo)-2-naphthylcarboxylic acid | 15850 | Red |
| 1-(2-Sulfo-4-methyl-5-chloro-1-phenylazo)-2-hydroxy- | 15865 | Red |
| naphthalene-3-carboxylic acid | | |
| 1-(2-Sulfo-1-naphthylazo)-2-hydroxynaphthalene-3-carboxylic | 15880 | Red |
| acid | | |
| 1-(3-Sulfo-1-phenylazo)-2-naphthol-6-sulfonic acid | 15980 | Orange |
| 1-(4-Sulfo-1-phenylazo)-2-naphthol-6-sulfonic acid | 15985 | Yellow |
| Allura Red | 16035 | Red |
| 1-(4-Sulfo-1-naphthylazo)-2-naphthol-3,6-disulfonic acid | 16185 | Red |
| Acid Orange 10 | 16230 | Orange |
| 1-(4-Sulfo-1-naphthylazo)-2-naphthol-6,8-disulfonic acid | 16255 | Red |
| 1-(4-Sulfo-1-naphthylazo)-2-naphthol-3,6,8-trisulfonic acid | 16290 | Red |
| 8-Amino-2-phenylazo-1-naphthol-3,6-disulfonic acid | 17200 | Red |
| Acid Red 1 | 18050 | Red |
| Acid Red 155 | 18130 | Red |
| Acid Yellow 121 | 18690 | Yellow |
| Acid Red 180 | 18736 | Red |
| Acid Yellow 11 | 18820 | Yellow |
| Acid Yellow 17 | 18965 | Yellow |
| 4-(4-Sulfo-1-phenylazo)-1-(4-sulfophenyl)-5-hydroxy- | 19140 | Yellow |
| pyrazolone-3-carboxylic acid | | |
| Pigment Yellow 16 | 20040 | Yellow |
| 2,6-(4′-Sulfo-2″,4″-dimethyl)bisphenylazo)-1,3-dihydroxy- | 20170 | Orange |
| benzene | | |
| Acid Black 1 | 20470 | Black |
| Pigment Yellow 13 | 21100 | Yellow |
| Pigment Yellow 83 | 21108 | Yellow |
| Solvent Yellow | 21230 | Yellow |
| Acid Red 163 | 24790 | Red |
| Acid Red 73 | 27290 | Red |
| 2-[4′-(4″-Sulfo-1″-phenylazo)-7′-sulfo-1′-naphthylazo]-1- | 27755 | Black |
| hydroxy-7-aminonaphthalene-3,6-disulfonic acid | | |
| 4-[4″-Sulfo-1″-phenylazo)-7′-sulfo-1′-naphthylazo]-1-hydroxy- | 28440 | Black |
| 8-acetylaminonaphthalene-3,5-disulfonic acid | | |
| Direct Orange 34, 39, 44, 46, 60 | 40215 | Orange |
| Food Yellow | 40800 | Orange |
| trans-β-Apo-8′-carotene aldehyde (C₃₀) | 40820 | Orange |
| trans-Apo-8′-carotinic acid (C₃₀) ethyl ester | 40850 | Orange |
| Canthaxanthine | 40850 | Orange |
| Acid Blue 1 | 42045 | Blue |
| 2,4-disulfo-5-hydroxy-4′-4″-bis(diethylamino)triphenylcarbinol | 42051 | Blue |
| 4-[(4-N-Ethyl-p-sulfobenzylamino)phenyl-(4-hydroxy-2-sulfo- | 42053 | Green |
| phenyl)(methylene)-1-(N-ethyl-N-p-sulfobenzyl)-2,5-cyclo- | | |
| hexadienimine] | | |
| Acid Blue 7 | 42080 | Blue |
| (N-Ethyl-p-sulfobenzylamino)phenyl-(2-sulfophenyl)methylene- | 42090 | Blue |
| (N-ethyl-N-p-sulfobenzyl)-Δ^{2,5}-cyclohexadienimine | | |
| Acid Green 9 | 42100 | Green |
| Diethyldisulfobenzyldi-4-amino-2-chlorodi-2-methylfuchsonimmonium | 42170 | Green |
| Basic Violet 14 | 42510 | Violet |
| Basic Violet 2 | 42520 | Violet |
| 2′-Methyl-4′-(N-ethyl-N-m-sulfobenzyl)amino-4″-(N-diethyl)- | 42735 | Blue |
| amino-2-methyl-N-ethyl-N-m-sulfobenzylfuchsonimmonium | | |
| 4′-(N-dimethyl)amino-4″-(N-phenyl)aminonaphtho-N- | 44045 | Blue |
| dimethylfuchsonimmonium | | |
| 2-Hydroxy-3,6-disulfo-4,4′-bisdimethylaminonaphthofuchsonimmonium | 44090 | Green |
| Acid Red 52 | 45100 | Red |
| 3-(2′-Methylphenylamino)-6-(2′-methyl-4′-sulfophenylamino)-9- | 45190 | Violet |
| (2″-carboxyphenyl)xanthenium salt | | |
| Acid Red 50 | 45220 | Red |
| Phenyl-2-oxyfluorone-2-carboxylic acid | 45350 | Yellow |
| 4,5-dibromofluorescein | 45370 | Orange |
| 2,4,5,7-Tetrabromofluorescein | 45380 | Red |
| Solvent Dye | 45396 | Orange |
| Acid Red 98 | 45405 | Red |
| 3′,4′,5′,6′-Tetrachloro-2,4,5,7-tetrabromofluorescein | 45410 | Red |
| 4,5-diiodofluorescein | 45425 | Red |
| 2,4,5,7-Tetraiodofluorescein | 45430 | Red |
| Quinophthalone | 47000 | Yellow |
| Quinophthalonedisulfonic acid | 47005 | Yellow |
| Acid Violet 50 | 50325 | Violet |
| Acid Black 2 | 50420 | Black |
| Pigment Violet 23 | 51319 | Violet |
| 1,2-dioxyanthraquinone, calcium-aluminium complex | 58000 | Red |
| 3-Oxypyrene-5,8,10-sulfonic acid | 59040 | Green |
| 1-Hydroxy-4-N-phenylaminoanthraquinone | 60724 | Violet |
| 1-Hydroxy-4-(4′-methylphenylamino)anthraquinone | 60725 | Violet |
| Acid Violet 23 | 60730 | Violet |
| 1,4-di(4′-methylphenylamino)anthraquinone | 61565 | Green |
| 1,4-Bis(o-sulfo-p-toluidino)anthraquinone | 61570 | Green |
| Acid Blue 80 | 61585 | Blue |
| Acid Blue 62 | 62045 | Blue |
| N,N′-dihydro-1,2,1′,2′-anthraquinonazine | 69800 | Blue |
| Vat Blue 6; Pigment Blue 64 | 69825 | Blue |
| Vat Orange 7 | 71105 | Orange |
| Indigo | 73000 | Blue |
| Indigodisulfonic acid | 73015 | Blue |
| 4,4′-dimethyl-6,6′-dichlorothioindigo | 73360 | Red |
| 5,5′-dichloro-7,7′-dimethylthioindigo | 73385 | Violet |
| Quinacridone Violet 19 | 73900 | Violet |
| Pigment Red 122 | 73915 | Red |
| Pigment Blue 16 | 74100 | Blue |
| Phthalocyanine | 74160 | Blue |
| Direct Blue 86 | 74180 | Blue |
| chlorinated phthalocyanine | 74260 | Green |
| Natural Yellow 6, 19; Natural Red 1 | 75100 | Yellow |
| Bixin, Nor-Bixin | 75120 | Orange |
| Lycopene | 75125 | Yellow |
| trans-alpha-, -beta- or -gamma-Carotene | 75130 | Orange |
| Keto and/or hydroxyl derivatives of carotene | 75135 | Yellow |
| Guanine or pearlescent agent | 75170 | White |
| 1,7-Bis(4-hydroxy-3-methoxyphenyl)-1,6-heptadiene-3,5-dione | 75300 | Yellow |
| Complex salt (Na, Al, Ca) of carminic acid | 75470 | Red |
| chlorophyll a and b; copper compounds of chlorophylls and | 75810 | Green |
| chlorophyllines | | |
| Aluminium | 77000 | White |
| Aluminium hydroxide | 77002 | White |
| Water-containing aluminium silicates | 77004 | White |
| Ultramarine | 77007 | Blue |
| Pigment Red 101 and 102 | 77015 | Red |
| Barium sulfate | 77120 | White |
| Bismuth oxychloride and mixtures thereof with mica | 77163 | White |
| Calcium carbonate | 77220 | White |
| Calcium sulfate | 77231 | White |
| Carbon | 77266 | Black |
| Pigment Black 9 | 77267 | Black |
| Carbo medicinalis vegetabilis | 77268:1 | Black |
| Chromium oxide | 77288 | Green |
| Chromium oxide, water-containing | 77278 | Green |
| Pigment Blue 28, Pigment Green 14 | 77346 | Green |
| Pigment Metal 2 | 77400 | Brown |
| Gold | 77480 | Brown |
| Iron oxides and hydroxides | 77489 | Orange |
| Iron oxide | 77491 | Red |
| Iron oxide hydrate | 77492 | Yellow |
| Iron oxide | 77499 | Black |
| Mixtures of iron(II) and iron(III) hexacyanoferrate | 77510 | Blue |
| Pigment White 18 | 77713 | White |
| Manganese ammonium diphosphate | 77742 | Violet |
| Manganese phosphate; Mn₃(PO₄)₂•7H₂O | 77745 | Red |
| Silver | 77820 | White |
| Titanium dioxide and mixtures thereof with mica | 77891 | White |
| Zinc oxide | 77947 | White |
| 6,7-dimethyl-9-(1′-D-ribityl)isoalloxazine, lactoflavin | | Yellow |
| Sugar dye | | Brown |
| Capsanthin, capsorubin | | Orange |
| Betanin | | Red |
| Benzopyrylium salts, anthocyans | | Red |
| Aluminium, zinc, magnesium and calcium stearate | | White |
| bromothymol Blue | | Blue |

It may furthermore be favourable to select, as dye, one or more sub-stances from the following group:

2,4-dihydroxyazobenzene, 1-(2′-chloro-4′-nitro-1′-phenylazo)-2-hydroxynaphthalene, Ceres Red, 2-(4-sulfo-1-naphthylazo)-1-naphthol-4-sulfonic acid, the calcium salt of 2-hydroxy-1,2′-azonaphthalene-1′-sulfonic acid, the calcium and barium salts of 1-(2-sulfo-4-methyl-1-phenylazo)-2-naphthylcarboxylic acid, the calcium salt of 1-(2-sulfo-1-naphthylazo)-2-hydroxynaphthalene-3-carboxylic acid, the aluminium salt of 1-(4-sulfo-1-phenylazo)-2-naphthol-6-sulfonic acid, the aluminium salt of 1-(4-sulfo-1-naphthylazo)-2-naphthol-3,6-disulfonic acid, 1-(4-sulfo-1-naphthylazo)-2-naphthol-6,8-disulfonic acid, the aluminium salt of 4-(4-sulfo-1-phenylazo)-2-(4-sulfophenyl)-5-hydroxypyrazolone-3-carboxylic acid, the aluminium and zirconium salts of 4,5-dibromofluorescein, the aluminium and zirconium salts of 2,4,5,7-tetrabromofluorescein, 3′,4′,5′,6′-tetrachloro-2,4,5,7-tetrabromofluorescein and its aluminium salt, the aluminium salt of 2,4,5,7-tetraiodofluorescein, the aluminium salt of quinophthalonedisulfonic acid, the aluminium salt of indigodisulfonic acid, red and black iron oxide (CIN: 77 491 (red) and 77 499 (black)), iron oxide hydrate (CIN: 77492), manganese ammonium diphosphate and titanium dioxide.

Also advantageous are oil-soluble natural dyes, such as, for example, paprika extract, β-carotene or cochineal.

Also advantageous for the purposes of the present invention are gel creams comprising pearlescent pigments. Particular preference is given to the types of pearlescent pigment listed below:
1. Natural pearlescent pigments, such as, for example,
   a) “pearl essence” (guanine/hypoxanthine mixed crystals from fish scales) and
   b) “mother-of-pearl” (ground mussel shells)
2. Monocrystalline pearlescent pigments, such as, for example, bismuth oxychloride (BiOCl)
3. Layered substrate pigments: for example mica/metal oxide

The basis for pearlescent pigments is formed by, for example, pulverulent pigments or castor oil dispersions of bismuth oxychloride and/or titanium dioxide as well as bismuth oxychloride and/or titanium dioxide on mica. The lustre pigment listed under CIN 77163, for example, is particularly advantageous.

Also advantageous are, for example, the following pearlescent pigment types based on mica/metal oxide:

| Group | Coating/layer thickness | Colour |
|---|---|---|
| Silver-white pearlescent | TiO₂: 40-60 nm | Silver |
| pigments | | |
| Interference pigments | TiO₂: 60-80 nm | Yellow |
| | TiO₂: 80-100 nm | Red |
| | TiO₂: 100-140 nm | Blue |
| | TiO₂: 120-160 nm | Green |
| Coloured lustre pigments | Fe₂O₃ | Bronze |
| | Fe₂O₃ | Copper |
| | Fe₂O₃ | Red |
| | Fe₂O₃ | Red-violet |
| | Fe₂O₃ | Red-green |
| | Fe₂O₃ | Black |
| Combination pigments | TiO₂/Fe₂O₃ | Gold shades |
| | TiO₂/Cr₂O₃ | Green |
| | TiO₂/Berlin Blue | Dark blue |

Particular preference is given to, for example, the pearlescent pigments available from Merck KGaA, Darmstadt, under the trade names Timiron®, Colorona® or Dichrona®.

The list of the said pearlescent pigments is of course not intended to be limiting. Pearlescent pigments which are advantageous for the purposes of the present invention can be obtained by numerous routes known per se. For example, other substrates apart from mica can also be coated with further metal oxides, such as, for example, silica and the like. For example, TiO₂— and Fe₂O₃-coated SiO₂ particles (“Ronasphere” grades), which are marketed by Merck KGaA, Darmstadt, and are particularly suitable for the optical reduction of fine wrinkles, are advantageous.

It may additionally be advantageous to completely omit a substrate such as mica. Particular preference is given to pearlescent pigments prepared using SiO₂. Such pigments, which may additionally also have goniochromatic effects, are available, for example, from BASF AG, Ludwigshafen, under the trade name Sicopearl® Fantastico.

It may also be advantageous to employ Engelhard/Mearl pigments based on calcium sodium borosilicate coated with titanium dioxide. These are available under the name Reflecks®. Due to their particle size of 40-80 μm, they have a glitter effect in addition to the colour.

Also particularly advantageous are effect pigments available from Flora Tech under the trade name Metasomes® Standard/Glitter in various colours (yellow, red, green and blue). The glitter particles here are in the form of mixtures with various assistants and dyes (such as, for example, the dyes with the colour index (CI) numbers 19140, 77007, 77289 and 77491).

The dyes and pigments can be in individual form or in the form of a mixture and mutually coated with one another, with different colour effects generally being caused by different coating thicknesses. The total amount of dyes and colouring pigments is advantageously selected from the range from, for example, 0.1% by weight to 30% by weight, preferably from 0.5 to 15% by weight, in particular from 1.0 to 10% by weight, in each case based on the total weight of the compositions.

All compounds or components which can be used in the compositions are either known and commercially available or can be synthesised by known processes. Any desired conventional carriers, assistants and optionally further active ingredients may be added to the composition.

Preferred assistants originate from the group consisting of preservatives, antioxidants, stabilisers, solubilisers, vitamins, colorants and odour improvers.

Solutions and emulsions may comprise the customary carriers, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

In a preferred embodiment, the compositions according to the invention comprise hydrophilic surfactants.

The hydrophilic surfactants are preferably selected from the group consisting of the alkylglucosides, acyl lactylates, betaines and coconut amphoacetates. The alkylglucosides are themselves advantageously selected from the group consisting of the alkylglucosides which are distinguished by the structural formula

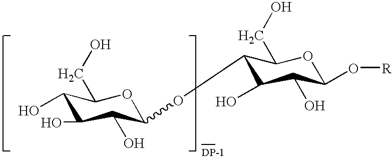

where R is a branched or unbranched alkyl radical having from 4 to 24 carbon atoms, and where D̅P̅ denotes a mean degree of glucosylation of up to 2.

The value D̅P̅ represents the degree of glucosidation of the alkylglucosides used in accordance with the invention and is defined as

$$\overline{DP} = \frac{{p}_{1}}{100} \cdot 1 + \frac{{p}_{2}}{100} \cdot 2 + \frac{{p}_{3}}{100} \cdot 3 + … = \sum \frac{{p}_{i}}{100} \cdot i$$

in which p₁, p₂, p₃ . . . pᵢ represent the proportion of mono-, di-, tri- . . . i-fold glucosylated products in percent by weight. Products which are advantageously selected in accordance with the invention are those having degrees of glucosylation of 1-2, particularly advantageously of from 1.1 to 1.5, very particularly advantageously of 1.2-1.4, in particular of 1.3.

The value DP takes into account the fact that alkylglucosides are, as a consequence of their preparation, generally mixtures of mono- and oligoglucosides. A relatively high content of monoglucosides, typically in the order of 40-70% by weight, is advantageous in accordance with the invention.

Alkylglycosides which are particularly advantageously used for the purposes of the invention are selected from the group consisting of octyl glucopyranoside, nonyl glucopyranoside, decyl glucopyranoside, undecyl glucopyranoside, dodecyl glucopyranoside, tetradecyl glucopyranoside and hexadecyl glucopyranoside.

It is likewise advantageous to employ natural or synthetic raw materials and assistants or mixtures which are distinguished by an effective content of the active ingredients used in accordance with the invention, for example Plantaren® 1200 (Henkel KGaA), Oramix® NS 10 (Seppic).

The acyllactylates are themselves advantageously selected from the group consisting of the substances which are distinguished by the structural formula

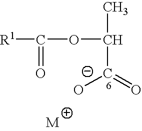

where R¹ denotes a branched or unbranched alkyl radical having from 1 to 30 carbon atoms, and M⁺ is selected from the group consisting of the alkali metal ions and the group consisting of ammonium ions which are substituted by one or more alkyl and/or one or more hydroxyalkyl radicals, or corresponds to half an equivalent of an alkaline earth metal ion.

For example, sodium isostearyl lactylate, for example the product Pathionic® ISL from the American Ingredients Company, is advantageous.

The betaines are advantageously selected from the group consisting of the substances which are distinguished by the structural formula

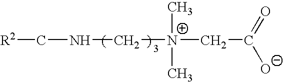

where R² denotes a branched or unbranched alkyl radical having from 1 to 30 carbon atoms.

R² particularly advantageously denotes a branched or unbranched alkyl radical having from 6 to 12 carbon atoms.

For example, capramidopropylbetaine, for example the product Tego® Betain 810 from Th. Goldschmidt AG, is advantageous.

A coconut amphoacetate which is advantageous for the purposes of the invention is, for example, sodium coconut amphoacetate, as available under the name Miranol® Ultra C32 from Miranol Chemical Corp.

The compositions according to the invention are advantageously characterised in that the hydrophilic surfactant(s) is (are) present in concentrations of 0.01-20% by weight, preferably 0.05-10% by weight, particularly preferably 0.1-5% by weight, in each case based on the total weight of the composition.

For use, the cosmetic and dermatological compositions according to the invention are applied in sufficient amount to the skin and/or hair in the usual manner for cosmetics.

The composition may comprise cosmetic adjuvants which are usually used in this type of composition, such as, for example, thickeners, softeners, moisturisers, surfactants, emulsifiers, preservatives, antifoams, perfumes, waxes, lanolin, propellants, dyes and/or pigments which colour the composition itself or the skin, and other ingredients usually used in cosmetics.

The dispersant or solubiliser used can be an oil, wax or other fatty substance, a lower monoalcohol or lower polyol or mixtures thereof. Particularly preferred monoalcohols or polyols include ethanol, isopropanol, propylene glycol, glycerol and sorbitol.

A preferred embodiment of the invention is an emulsion in the form of a protective cream or milk which comprises, for example, fatty alcohols, fatty acids, fatty acid esters, in particular triglycerides of fatty acids, lanolin, natural and synthetic oils or waxes and emulsifiers in the presence of water.

If a composition is formulated as an aerosol, the customary propellants, such as alkanes, fluoroalkanes and chlorofluoroalkanes, are generally used.

The cosmetic composition may also be used to protect the hair against photochemical damage in order to prevent changes of colour shade, bleaching or damage of a mechanical nature. In this case, a suitable formulation is in the form of a rinse-out shampoo, lotion, gel or emulsion, the composition in question being applied before or after shampooing, before or after colouring or bleaching or before or after permanent waving. It is also possible to select a composition in the form of a lotion or gel for styling or treating the hair, in the form of a lotion or gel for brushing or blow-waving the hair, in the form of a hair lacquer, permanent waving composition, colorant or bleach for the hair. The composition having light-protection properties may comprise various adjuvants used in this type of composition, such as surfactants, thickeners, polymers, softeners, preservatives, foam stabilisers, electrolytes, organic solvents, silicone derivatives, oils, waxes, antigrease agents, dyes and/or pigments which colour the composition itself or the hair, or other ingredients usually used for hair care.

The compositions according to the invention can be prepared here with the aid of techniques which are well known to the person skilled in the art. The substances according to the invention can be incorporated directly into cosmetic compositions without further preparatory measures.

The compositions according to the invention may advantageously, as already described above, comprise further UV filter substances, where the total amount of the filter substances is, for example, from 0.1% by weight to 30% by weight, preferably from 0.5 to 10% by weight, in particular from 1 to 6% by weight, based on the total weight of the compositions.

Furthermore, the compositions according to the invention can also be used as pharmaceutical compositions for the preventative treatment of inflammation and allergies of the skin and also in certain cases for the prevention of certain types of cancer. The pharmaceutical compositions according to the invention can be administered orally or topically.

The compositions according to the invention can be prepared with the aid of techniques which are well known to the person skilled in the art. Even without further comments, it is assumed that a person skilled in the art will be able to utilise the above description in its broadest scope. The preferred embodiments should therefore merely be regarded as descriptive disclosure which is absolutely not limiting in any way. The following examples are intended to illustrate the present invention without restricting it. All amount data, proportions and percentages are, unless stated otherwise, based on the weight and the total amount or total weight of the compositions. The complete disclosure content of all applications and publications mentioned above and below is incorporated into this application by way of reference.

### EXAMPLES

For the naming of compounds, the INCl names of the commercially available compounds are indicated as far as possible.

### Example 1

### Production of a silica capsule containing Diethylamino Hydroxybenzoyl Hexyl Benzoate

A solution of 33.5 g of Uvinul A Plus, 66.5 g of Dibutyl Adipate and 17.7 g of tetraethyl orthosilicate is emulsified in a surfactant solution (58.2 g of deionised water and 0.69 g of cetyltrimethylammonium chloride) with cooling with the aid of an emulsification tool (Ultra Turrax). The finished emulsion is added with stirring to water containing hydrochloric acid. The resultant mixture is stirred at room temperature for 24 h and subsequently left to stand again without stirring for 24 h. The ethanol formed on hydrolysis of the alkylsilane is then partially removed by distillation. The pH of the residue is adjusted to 3.8-4.0 using Na citrate solution, and the mixture is made up with deionised water.

The active-compound content of the suspension is 50% by weight.

The mixture can be incorporated into the cosmetic composition in this form.

The silica capsules containing Uvinul A Plus are isolated by spray drying, giving a white powder.

### Example 2

### Production of a Silica Capsule Containing Diethylamino Hydroxybenzoyl Hexyl Benzoate and Ethylhexyl Methoxycinnamate

A solution of 36 g of hexyl 2-(4-diethylamino-2-hydroxybenzoyl)benzoate (Uvinul® A Plus; BASF AG), 64 g of ethylhexyl methoxycinnamate (for example Eusolex® 2292; Merck) and 26.5 g of tetraethyl orthosilicate is emulsified in a surfactant solution (58.2 g of deionised water and 0.69 g of cetyltrimethylammonium chloride) with cooling with the aid of an emulsification tool (Ultra Turrax). The finished emulsion is added with stirring to water containing hydrochloric acid. The resultant mixture is stirred at room temperature for at least 24 h and subsequently left to stand again without stirring for at least 24 h. The ethanol formed on hydrolysis of the alkylsilane is then partially removed by distillation. The pH of the residue is adjusted to 3.8-4.0 using Na citrate solution. 4 g of PVP solution comprising 50% by weight of polyvinylpyrrolidone are added, and the mixture is made up to 200 g with deionised water. The active-compound content of the suspension is 50% by weight.

### Example 3

### Production of a Silica Capsule Containing Diethylamino Hydroxybenzoyl Hexyl Benzoate and 2-ethylhexyl Salicylate (Eusolex® OS)

A solution of 25 g of hexyl 2-(4-diethylamino-2-hydroxybenzoyl)benzoate (Uvinul® A Plus; BASF AG), 75 g of 2-ethylhexyl salicylate (Eusolex® OS; Merck) and 17.7 g of tetraethyl orthosilicate is emulsified in a surfactant solution (58.2 g of deionised water and 0.69 g of cetyltrimethylammonium chloride) with cooling with the aid of an emulsification tool (Ultra Turrax). The finished emulsion is added with stirring to water containing hydrochloric acid. The resultant mixture is stirred at room temperature for 24 h and subsequently left to stand again without stirring for 24 h. The ethanol formed on hydrolysis of the alkylsilane is then partially removed by distillation. The pH of the residue is adjusted to 3.8-4.0 using Na citrate solution. 4 g of PVP solution comprising 50% by weight of polyvinylpyrrolidone are added, and the mixture is made up to 200 g with deionised water. The active-compound content of the suspension is 50% by weight.

### Example 3a

The production of a silica capsule containing Diethylamino Hydroxybenzoyl Hexyl Benzoate and 3,3,5-trimethylcyclohexyl salicylate (for example Eusolex® HMS) is carried out analogously to Example 3.

### Example 4

### Production of a Silica Capsule Containing Diethylamino Hydroxybenzoyl Hexyl Benzoate and 2-ethylhexyl 2-cyano-3,3-diphenylacrylate

A solution of 64 g of Diethylamino Hydroxybenzoyl) Hexyl Benzoate (Uvinul® A Plus; BASF AG), 36 g of 2-ethylhexyl 2-cyano-3,3-diphenylacrylate (Uvinul® A Plus B; BASF) and 26.5 g of tetraethyl orthosilicate is emulsified in a surfactant solution (58.2 g of deionised water and 0.69 g of cetyltrimethylammonium chloride) with cooling with the aid of an emulsification tool (Ultra Turrax). The finished emulsion is added with stirring to water containing hydrochloric acid. The resultant mixture is stirred at room temperature for at least 24 h and subsequently left to stand again without stirring for at least 24 h. The ethanol formed on hydrolysis of the alkylsilane is then partially removed by distillation. The pH of the residue is adjusted to 3.8-4.0 using Na citrate solution. 4 g of PVP solution comprising 50% by weight of polyvinylpyrrolidone are added, and the mixture is made up to 200 g with deionised water. The active-compound content of the suspension is 50% by weight.

### Example 5

### Sunscreen for Sensitive Skin (W/O) In Vivo SPF 46 (International SPF Method; 10 Volunteers)

| Ingredient | [%] |
|---|---|
| TITANIUM DIOXIDE, ALUMINA, SIMETHICONE | 15.00 |
| POLYGLYCERYL-2 | 4.00 |
| DIPOLYHYDROXYSTEARATE | |
| POLYGLYCERYL-3 DIISOSTEARATE | 2.00 |
| DICAPRYLYL CARBONATE | 11.00 |
| COCOGLYCERIDES | 10.00 |
| CERA ALBA (BEESWAX) | 2.50 |
| ISOSTEARIC ACID | 1.00 |
| ZINC STEARATE | 1.00 |
| TOCOPHEROL | 2.00 |
| PROPYLPARABEN | 0.05 |
| Capsule according to Ex. 1 | 10.00 |
| GLYCERIN | 5.00 |
| MAGNESIUM SULFATE | 1.00 |
| METHYLPARABEN | 0.15 |
| AQUA (WATER) | 34.80 |
| BISABOLOL | 0.50 |

### Example 6

### Composition for Lip Care

| (% by weight) | |
|---|---|
| to 100 | Eucerinum Anhydricum |
| 10.00 | Glycerin |
| 10.00 | Titanium Dioxide, micronised |
| 5.00 | capsules according to Example 1 |
| 8.00 | Octyl Methoxycinnamate |
| 5.00 | Zinc Oxide |
| 4.00 | Castor Oil |
| 4.00 | Pentaerythritol |
| | Stearate/Caprate/Caprylate Adipate |
| 3.00 | Glyceryl Stearate SE |
| 2.00 | Beeswax |
| 2.00 | Microcrystalline Wax |
| 2.00 | Quaternium-18 Bentonite |
| 1.50 | PEG-45/Dodecyl Glycol Copolymer |

### Example 7

### Composition for Lip Care

| (% by weight) | |
|---|---|
| to 100 | Eucerinum Anhydricum |
| 10.00 | Glycerin |
| 10.00 | Titanium Dioxide, micronised |
| 5.00 | capsules according to Example 2 |
| 8.00 | Octyl Methoxycinnamate |
| 5.00 | Zinc Oxide |
| 4.00 | Castor Oil |
| 4.00 | Pentaerythritol |
| | Stearate/Caprate/Caprylate Adipate |
| 3.00 | Glyceryl Stearate SE |
| 2.00 | Beeswax |
| 2.00 | Microcrystalline Wax |
| 2.00 | Quaternium-18 Bentonite |
| 1.50 | PEG-45/Dodecyl Glycol Copolymer |

### Example 8

### Composition for Sunblocker with Micropigments

| (% by weight) | |
|---|---|
| to 100 | water |
| 10.00 | Octyl Methoxycinnamate |
| 6.00 | PEG-7 Hydrogenated Castor Oil |
| 6.00 | Titanium Dioxide, micronised |
| 5.00 | capsules according to Example 3 |
| 5.00 | Mineral Oil |
| 5.00 | Isoamyl p-Methoxycinnamate |
| 5.00 | Propylene Glycol |
| 3.00 | Jojoba Oil |
| 3.00 | 4-Methylbenzylidene Camphor |
| 2.00 | PEG-45/Dodecyl Glycol Copolymer |
| 1.00 | Dimethicone |
| 0.50 | PEG-40 Hydrogenated Castor Oil |
| 0.50 | Tocopheryl Acetate |
| 0.50 | Phenoxyethanol |
| 0.20 | EDTA |

### Example 9

### Composition for Sunblocker with Micropigments

| (% by weight) | |
|---|---|
| to 100 | water |
| 10.00 | Octyl Methoxycinnamate |
| 6.00 | PEG-7-Hydrogenated Castor Oil |
| 6.00 | Titanium Dioxide, micronised |
| 5.00 | capsules according to Example 2 |
| 5.00 | Mineral Oil |
| 5.00 | Isoamyl p-Methoxycinnamate |
| 5.00 | Propylene Glycol |
| 3.00 | Jojoba Oil |
| 3.00 | 4-Methylbenzylidene Camphor |
| 2.00 | PEG-45/Dodecyl Glycol Copolymer |
| 1.00 | Dimethicone |
| 0.50 | PEG-40 Hydrogenated Castor Oil |
| 0.50 | Tocopheryl Acetate |
| 0.50 | Phenoxyethanol |
| 0.20 | EDTA |

### Example 10

### Fat-Free Gel

| (% by weight) | |
|---|---|
| to 100 | water |
| 8.00 | Octyl Methoxycinnamate |
| 7.00 | Titanium Dioxide, micronised |
| 5.00 | capsules according to Example 4 |
| 5.00 | Glycerin |
| 5.00 | PEG-25 PABA |
| 1.00 | 4-Methylbenzylidene Camphor |
| 0.40 | Acrylate C10-C30 Alkyl Acrylate Crosspolymer |
| 0.30 | Imidazolidinyl Urea |
| 0.25 | Hydroxyethyl Cellulose |
| 0.25 | Sodium Methylparaben |
| 0.20 | Disodium EDTA |
| 0.15 | Fragrance |
| 0.15 | Sodium Propylparaben |
| 0.10 | Sodium Hydroxide |

### Example 11

### Fat-Free Gel

| (% by weight) | |
|---|---|
| to 100 | water |
| 8.00 | Octyl Methoxycinnamate |
| 7.00 | Titanium Dioxide, micronised |
| 5.00 | capsules according to Example 1 |
| 5.00 | Glycerin |
| 5.00 | PEG-25 PABA |
| 1.00 | 4-Methylbenzylidene Camphor |
| 0.40 | Acrylate C10-C30 Alkyl Acrylate Crosspolymer |
| 0.30 | Imidazolidinyl Urea |
| 0.25 | Hydroxyethyl Cellulose |
| 0.25 | Sodium Methylparaben |
| 0.20 | Disodium EDTA |
| 0.15 | Fragrance |
| 0.15 | Sodium Propylparaben |
| 0.10 | Sodium Hydroxide |

### Example 12

### Sun Cream (LPF 20)

| (% by weight) | |
|---|---|
| to 100 | water |
| 8.00 | Octyl Methoxycinnamate |
| 8.00 | Titanium Dioxide, micronised |
| 6.00 | PEG-7 Hydrogenated Castor Oil |
| 5.00 | capsules according to Example 2 |
| 6.00 | Mineral Oil |
| 5.00 | Zinc Oxide |
| 5.00 | Isopropyl Palmitate |
| 0.30 | Imidazolidinyl Urea |
| 3.00 | Jojoba Oil |
| 2.00 | PEG-45/Dodecyl Glycol Copolymer |
| 1.00 | 4-Methylbenzylidene Camphor |
| 0.60 | Magnesium Stearate |
| 0.50 | Tocopheryl Acetate |
| 0.25 | Methylparaben |
| 0.20 | Disodium EDTA |
| 0.15 | Propylparaben |

### Example 13

### Sun Cream (LSF 20)

| (% by weight) | |
|---|---|
| to 100 | water |
| 8.00 | Octyl Methoxycinnamate |
| 8.00 | Titanium Dioxide, micronised |
| 6.00 | PEG-7 Hydrogenated Castor Oil |
| 5.00 | capsules according to Example 1 |
| 6.00 | Mineral Oil |
| 5.00 | Zinc Oxide |
| 5.00 | Isopropyl Palmitate |
| 0.30 | Imidazolidinyl Urea |
| 3.00 | Jojoba Oil |
| 2.00 | PEG-45/Dodecyl Glycol Copolymer |
| 1.00 | 4-Methylbenzylidene Camphor |
| 0.60 | Magnesium Stearate |
| 0.50 | Tocopheryl Acetate |
| 0.25 | Methylparaben |
| 0.20 | Disodium EDTA |
| 0.15 | Propylparaben |

### Example 14

### Water-Resistant Sun Cream

| (% by weight) | |
|---|---|
| to 100 | water |
| 8.00 | Octyl Methoxycinnamate |
| 5.00 | PEG-7 Hydrogenated Castor Oil |
| 5.00 | Propylene Glycol |
| 4.00 | Isopropyl Palmitate |
| 4.00 | Caprylic/Capric Triglyceride |
| 5.00 | capsules according to Example 4 |
| 4.00 | Glycerin |
| 3.00 | Jojoba Oil |
| 2.00 | 4-Methylbenzylidene Camphor |
| 2.00 | Titanium Dioxide, micronised |
| 1.50 | PEG-45/Dodecyl Glycol Copolymer |
| 1.50 | Dimethicone |
| 0.70 | Magnesium Sulfate |
| 0.50 | Magnesium Stearate |
| 0.15 | Fragrance |

### Example 15

### Water-Resistant Sun Cream

| (% by weight) | |
|---|---|
| to 100 | water |
| 8.00 | Octyl Methoxycinnamate |
| 5.00 | PEG-7-Hydrogenated Castor Oil |
| 5.00 | Propylene Glycol |
| 4.00 | Isopropyl Palmitate |
| 4.00 | Caprylic/Capric Triglyceride |
| 5.00 | capsules according to Example 3 |
| 4.00 | Glycerin |
| 3.00 | Jojoba Oil |
| 2.00 | 4-Methylbenzylidene Camphor |
| 2.00 | Titanium Dioxide, micronised |
| 1.50 | PEG-45/Dodecyl Glycol Copolymer |
| 1.50 | Dimethicone |
| 0.70 | Magnesium Sulfate |
| 0.50 | Magnesium Stearate |
| 0.15 | Fragrance |

### Example 16

### Sun Milk (LPF 6)

| (% by weight) | |
|---|---|
| to 100 | water |
| 10.00 | Mineral Oil |
| 6.00 | PEG-7 Hydrogenated Castor Oil |
| 5.00 | Isopropyl Palmitate |
| 3.50 | Octyl Methoxycinnamate |
| 5.00 | capsules according to Example 4 |
| 3.00 | Caprylic/Capric Triglyceride |
| 3.00 | Jojoba Oil |
| 2.00 | PEG-45/Dodecyl Glycol Copolymer |
| 0.70 | Magnesium Sulfate |
| 0.60 | Magnesium Stearate |
| 0.50 | Tocopheryl Acetate |
| 3.00 | Glycerin |
| 0.25 | Methylparaben |
| 0.15 | Propylparaben |
| 0.05 | Tocopherol |

### Example 17

### Sun Milk (LPF 6)

| (% by weight) | |
|---|---|
| to 100 | water |
| 10.00 | Mineral Oil |
| 6.00 | PEG-7 Hydrogenated Castor Oil |
| 5.00 | Isopropyl Palmitate |
| 3.50 | Octyl Methoxycinnamate |
| 5.00 | capsules according to Example 4 |
| 3.00 | Caprylic/Capric Triglyceride |
| 3.00 | Jojoba Oil |
| 2.00 | PEG-45/Dodecyl Glycol Copolymer |
| 0.70 | Magnesium Sulfate |
| 0.60 | Magnesium Stearate |
| 0.50 | Tocopheryl Acetate |
| 3.00 | Glycerin |
| 0.25 | Methylparaben |
| 0.15 | Propylparaben |
| 0.05 | Tocopherol |

### Example 18

### Sunscreen for Sensitive Skin (W/O) In Vivo SPF 46 (International SPF Method with 10 Volunteers)

| Ingredient | [%] |
|---|---|
| TITANIUM DIOXIDE, ALUMINA, SIMETHICONE | 15.00 |
| POLYGLYCERYL-2 | 4.00 |
| DIPOLYHYDROXYSTEARATE | |
| POLYGLYCERYL-3 DIISOSTEARATE | 2.00 |
| DICAPRYLYL CARBONATE | 11.00 |
| COCOGLYCERIDES | 10.00 |
| CERA ALBA (BEESWAX) | 2.50 |
| ISOSTEARIC ACID | 1.00 |
| ZINC STEARATE | 1.00 |
| TOCOPHEROL | 2.00 |
| PROPYLPARABEN | 0.05 |
| Capsules according to Ex. 4 | 10.00 |
| GLYCERIN | 5.00 |
| MAGNESIUM SULFATE | 1.00 |
| METHYLPARABEN | 0.15 |
| AQUA (WATER) | 34.80 |
| BISABOLOL | 0.50 |

### Example 19

### Care Cream In Vitro SPF (Diffey Method)=19±7

| Ingredient | [%] |
|---|---|
| TITANIUM DIOXIDE, ALUMINA, SIMETHICONE | 5.00 |
| CETEARYL ALCOHOL, CETEARYL GLUCOSIDE | 4.00 |
| CETEARYL ALCOHOL | 1.00 |
| SORBITAN STEARATE | 1.50 |
| ISOPROPYL PALMITATE | 1.00 |
| ISOHEXADECANE | 1.00 |
| GLYCINE SOJA (SOYBEAN OIL) | 0.50 |
| DIMETHICONE | 1.00 |
| UREA, DISODIUM PHOSPHATE, BIOTIN, CITRIC ACID | 1.00 |
| ALLANTOIN | 0.30 |
| GLYCERIN | 10.00 |
| PANTHENOL | 0.50 |
| AQUA (WATER) | 57.00 |
| XANTHAN GUM | 0.30 |
| Capsules according to Ex. 4 | 15.00 |
| PERFUME | 0.20 |
| PHENOXYETHANOL, BUTYLPARABEN, | 0.70 |
| ETHYLPARABEN, PROPYLPARABEN, METHYLPARABEN | |

### Example 20

### Eye-Care Cream

| Ingredient | [%] |
|---|---|
| TITANIUM DIOXIDE, ALUMINA, STEARIC ACID | 5.00 |
| POLYGLYCERYL-3 METHYLGLUCOSE DISTEARATE | 3.00 |
| GLYCERYL STEARATE | 2.00 |
| STEARYL ALCOHOL | 1.50 |
| CAPRYLIC/CAPRIC TRIGLYCERIDE | 5.00 |
| PERSEA GRATISSIMA (AVOCADO OIL) | 1.50 |
| ISOHEXADECANE | 8.00 |
| PROPYLPARABEN | 0.05 |
| SILICA, CI 77891 (TITANIUM DIOXIDE), CI 77491 | 5.00 |
| (IRON OXIDES) | |
| UREA, DISODIUM PHOSPHATE, BIOTIN, CITRIC ACID | 2.00 |
| GLYCERIN | 3.00 |
| METHYLPARABEN | 0.15 |
| AQUA (WATER) | 53.80 |
| Capsules according to Ex. 1 | 10.00 |
| PERFUME | q.s. |

### Example 21

### Foundation with an In Vitro SPF (Diffey) of 10.9/eryth. UVA-PF 4

| Ingredient | [%] |
|---|---|
| WAX | 5.00 |
| ISOPROPYL MYRISTATE | 2.00 |
| CETEARYL ALCOHOL | 1.00 |
| AQUA (WATER) | 70.45 |
| KAOLIN | 8.00 |
| Microna ® Matte Black (Merck) CI 77499 | 0.15 |
| (IRON OXIDES), MICA | |
| Microna ® Matte Red (Merck) CI 77491 | 0.50 |
| (IRON OXIDES), MICA | |
| Microna ® Matte Yellow (Merck) MICA, CI 77492 | 2.00 |
| (IRON OXIDES) | |
| Capsules according to Ex. 3 | 10.00 |
| PROPYLENE GLYCOL, DIAZOLIDINYL UREA, | 0.90 |
| METHYLPARABEN, PROPYLPARABEN | |

### Example 22

### Sun Cream Comprising Eusolex® UV-Pearls™ OMC In Vitro SPF (Diffey method) 21.5±5.7/eryth. UVA-PF 7.5±0.7

| Ingredient | [%] |
|---|---|
| CETYL PALMITATE, GLYCERYL STEARATE, | 6.00 |
| CETEARETH-20, CETEARETH-12, CETEARYL | |
| ALCOHOL | |
| CETEARYL ALCOHOL | 1.50 |
| DECYL OLEATE | 2.00 |
| TITANIUM DIOXIDE, ALUMINA, STEARIC ACID | 8.00 |
| ETHYLHEXYL STEARATE | 2.00 |
| CHITOSAN GLYCOLATE | 10.00 |
| AQUA (WATER) | 48.60 |
| Capsules according to Ex. 4 | 21.00 |
| PROPYLENE GLYCOL, DIAZOLIDINYL UREA, | 0.90 |
| METHYLPARABEN, PROPYLPARABEN | |

### Example 23

### Moisturising Fluid Comprising Eusolex® UV-Pearls OMC Oil-Free, with Soft Sheen, SPF (DIFFEY) 8.9±1.3, UVA-PF 3.2±0.3

| Ingredient | [%] |
|---|---|
| XANTHAN GUM | 1.50 |
| GLYCERIN | 20.00 |
| PPG-5-CETETH-20 | 1.00 |
| Capsules according to Ex. 4 | 20.00 |
| SODIUM PCA | 3.00 |
| Timiron ® Karat Gold MP-24 MICA, CI 77891 | 0.50 |
| (TITANIUM DIOXIDE), CI 77491 (IRON OXIDES) | |
| Timiron ® Sun Gold Sparkle MP-29MICA, CI 77891 | 0.50 |
| (TITANIUM DIOXIDE), CI 77491 (IRON OXIDES) | |
| AQUA (WATER) | 52.80 |
| PROPYLENE GLYCOL, DIAZOLIDINYL UREA, | 0.70 |
| METHYLPARABEN, PROPYLPARABEN | |

### Example 24

### Sunscreen Gel for Sensitive Skin

| Ingredient | [%] |
|---|---|
| AQUA (WATER) | 52.80 |
| TROMETHAMINE | 0.40 |
| BUTYL METHOXYDIBENZOYLMETHANE | 2.00 |
| DIBUTYL ADIPATE | 8.00 |
| C12-15 ALKYL BENZOATE | 6.50 |
| COCOGLYCERIDES | 3.00 |
| CETEARYL OCTANOATE | 2.00 |
| DIMETHICONE | 2.00 |
| BUXUS CHINENSIS (JOJOBA OIL) | 1.00 |
| TOCOPHERYL ACETATE | 0.50 |
| ACRYLATES/C10-30 ALKYL ACRYLATE | 0.20 |
| CROSSPOLYMER | 0.10 |
| CARBOMER | |
| PHENOXYETHANOL, BUTYLPARABEN, | 0.50 |
| ETHYLPARABEN, PROPYLPARABEN, | |
| METHYLPARABEN | |
| Capsules according to Ex. 4 | 10% |
| Capsules according to Ex. 3 | 11% |

### Example 25

### Moisturising Cream In Vivo SPF 12 (Colipa)

| Ingredient | [%] |
|---|---|
| CAPSULES ACCORDING TO EX. 4 | 9.20 |
| SILICA, CI 77891 (TITANIUM DIOXIDE), CI 77491 | 5.00 |
| (IRON OXIDES) | |
| ECTOIN | 0.20 |
| PROPYLENE GLYCOL | 3.00 |
| GLYCERIN | 3.00 |
| LAURETH-7, POLYACRYLAMIDE, | 2.50 |
| C13-14 ISOPARAFFIN | |
| PROPYLENE GLYCOL, DIAZOLIDINYL UREA, | 0.70 |
| METHYLPARABEN, PROPYLPARABEN | 75.60 |
| AQUA (WATER) | |

1. UV filter capsule containing at least one amino-substituted hydroxybenzophenone.

2. UV filter capsule according to claim 1, characterised in that the at least one amino-substituted hydroxybenzophenone derivative is selected from the compounds of the formula I 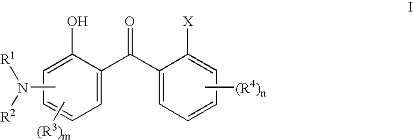
in which the variables, independently of one another, have the following meaning:
R¹ and R² hydrogen, C₁-C₂₀-alkyl, C₂-C₁₀-alkenyl, C₃-C₁₀-cycloalkyl, C₃-C₁₀-cycloalkenyl, where the substituents R¹ and R² can form a 5- or 6-membered ring together with the nitrogen atom to which they are bonded;
R³ and R⁴ C₁-C₂₀-alkyl, C₂-C₁₀-alkenyl, C₃-C₁₀-cycloalkyl, C₃-C₁₀-cycloalkenyl, C₁-C₁₂-alkoxy, C₁-C₂₀-alkoxy, C₁-C₂₀-alkoxycarbonyl, C₁-C₁₂-alkylamino, C₁-C₁₂-dialkylamino, aryl, heteroaryl, optionally substituted, water-solubilising substituents selected from the group consisting of a nitrile group, carboxylate, sulfonate or ammonium radicals;
X hydrogen, COOR⁵, CONR⁶R⁷;
R⁵ to R⁷ hydrogen, C₁-C₂₀-alkyl, C₂-C₁₀-alkenyl, C₃-C₁₀-cycloalkyl, C₃-C₁₀-cycloalkenyl, —(Y—O)ₒ-Z, aryl;
Y —(CH₂)₂—, —(CH₂)₄—, —CH(CH₃)—CH₂—;
Z —CH₂—CH₃, —CH₂—CH₂-CH₃, —CH₂—CH₂-CH₂—CH₃, —CH/(CH₃)—CH₃;
m 0 to 3;
n 0 to 4;
o 1 to 20.

3. UV filter capsule according to claim 1, characterised in that the capsule consists of 1 to 99% by weight of amino-substituted hydroxybenzophenone.

4. UV filter capsule according to claim 1, characterised in that the capsule contains a cosmetic oil, preferably an ester of saturated and/or unsaturated, branched and/or unbranched alkanecarboxylic acids having a chain length of 3 to 30 C atoms and saturated and/or unsaturated, branched and/or unbranched alkylene glycols having a chain length of 2 to 30 C atoms.

5. UV filter capsule according to claim 1, characterised in that the capsule contains (INCl names) Octocrylene, Ethylhexyl Methoxycinnamate, Dibutyl Adipate, C12-15 Alkyl Benzoate, C12-13 Alkyl Lactate, Propylene Glycol Dicaprylate/Dicaprate, Diethylhexyl Adipate, PEG-7 Glyceryl Cocoate, Caprylic/Capric Triglyceride, Ethylhexyl Ethylhexanoate, Isopropyl Alcohol, PPG-3 Myristyl Ether, D1-C12-13 Alkyl Tartrate, Ethanol, Hexyl Laurate, PEG-7 Hydrogenated Castor Oil, Di-C12-13 Alkyl Malate and/or Isopropyl Stearate.

6. UV filter capsule according to claim 1, characterised in that the capsule contains at least one UVB filter.

7. UV filter capsule according to claim 1, characterised in that the capsule contains hexyl 2-(4-diethylamino-2-hydroxybenzoyl)benzoate as amino-substituted hydroxybenzophenone.

8. UV filter capsule according to claim 1, characterised in that the capsule contains amino-substituted hydroxybenzophenone and UV-B filters in the percent by weight ratio 10:1 to 1:3, preferably in the percent by weight ratio 5:1 to 1:2.

9. UV filter capsule according to claim 1, characterised in that
the capsules contains 5 to 42% by weight of amino-substituted hydroxybenzophenone and 58 to 95% by weight of cinnamic acid derivative, or
the capsule contains 5 to 44% by weight of amino-substituted hydroxybenzophenone and 56 to 95% by weight of diphenylacrylate derivative, or
the capsule contains 50 to 90% by weight of amino-substituted hydroxybenzophenone and 10 to 50% by weight of salicylic acid derivative.

10. Formulation comprising at least one amino-substituted hydroxybenzophenone and at least one vehicle which is suitable for topical purposes, characterised in that at least part of the amino-substituted hydroxybenzophenones is in the form of a capsule according to claim 1.

11. Formulation according to claim 10, characterised in that it is an aqueous dispersion which preferably comprises 5 to 80% by weight of capsules.

12. A method of using a capsule according to claim 1 comprising preparing a cosmetic or dermatological composition having light-protection properties with said capsule.

13. A method according to claim 12, characterised in that the cosmetic or dermatological composition is an aqueous composition, in particular a gel, or an oil-in-water emulsion (O/W emulsion).

14. Process for the preparation of a cosmetic or dermatological composition having light-protection properties, characterised in that a capsule according to claim 1 is mixed with further ingredients.

15. Process for the preparation of a cosmetic or dermatological composition having light-protection properties, characterised in that the cosmetic or dermatological composition is an oil-in-water emulsion (O/W emulsion), and a formulation according to claim 10 is emulsified with an oil.

16. Composition comprising a formulation according to claim 10, characterised in that it is an aqueous composition, in particular a gel, or an emulsion, in particular an oil-in-water emulsion (O/W emulsion).

17. Emulsion according to claim 16, characterised in that the water phase is formed by said formulation.

18. Process for the production of UV filter capsules according to claim 1, characterised in that in step a), an oil-in-water emulsion of a hydrophobic solution comprising the sol-gel precursor, at least one amino-substituted hydroxybenzophenone and optionally at least one cosmetic oil and/or at least one further UVB filter is prepared in an aqueous solution, in step b), this emulsion from step a) is mixed with another aqueous solution in order to accelerate the condensation polymerisation reaction, optionally in step c), reaction products are separated off from the sol-gel precursor, and the UV filter capsules are isolated.
